SEQUENCE LISTING (2 sequences)

SEQ ID NO: 1 - protein, 107 aa, Homo sapiens
IEVMYPPPYLDNEKSNGTIIHVKGKHLCPSPLFPGPSKPFWVLVVVGGVLACYSLLVTVAFIIFWVRSKRSRLLHSDYMNMTPRRPGPTRKHYQPYAPPRDFAAYRS

SEQ ID NO: 2 - protein, 9 aa, Homo sapiens
IEVMYPPPY

(19) United States
(12) Patent Application Publication
(10) Pub. No.: US 2025/0236862 A1
(43) Pub. Date: Jul. 24, 2025

(54) METHODS AND COMPOSITIONS FOR HIGH-THROUGHPUT DISCOVERY OF PEPTIDE-MHC TARGETING BINDING PROTEINS

(71) Applicants: The General Hospital Corporation, Boston, MA (US); The Broad Institute, Inc., Cambridge, MA (US); Massachusetts Institute of Technology, Cambridge, MA (US)

(72) Inventors: Nir Hacohen, Boston, MA (US); Bingxu Liu, Cambridge, MA (US); Matthew Bakalar, Cambridge, MA (US); Xun Chen, Cambridge, MA (US)

(21) Appl. No.: 19/171,741

(22) Filed: Apr. 7, 2025

Related U.S. Application Data
(63) Continuation of application No. PCT/US2023/076267, filed on Oct. 6, 2023.
(60) Provisional application No. 63/414,303, filed on Oct. 7, 2022.

(51) Int. Cl. C12N 15/10; C07K 16/28

(52) U.S. Cl. CPC C12N 15/1041; C07K 16/2833; C07K 2317/565; C07K 2317/569; C07K 2317/92

(57) ABSTRACT

The present invention discloses methods and platforms for generating protein binding proteins with specificity for native peptide-MHC (pMHC) complexes. The pMHC binding proteins can be used in bi-specific antibodies or for generating CAR T cells capable of binding to peptides bound to specific MHC alleles.

### CROSS-REFERENCE TO RELATED APPLICATIONS

This application is a continuation application of PCT/US2023/076267, filed Oct. 6, 2023, which claims the benefit of U.S. Provisional Application No. 63/414,303,filed Oct. 7, 2022. The entire contents of the above-identified applications are hereby fully incorporated herein by reference.

### REFERENCE TO AN ELECTRONIC SEQUENCE LISTING

The contents of the electronic sequence listing (“BROD-5690US_ST26.xml”; Size is 2,918 bytes and it was created on Apr. 4, 2025) is herein incorporated by reference in its entirety.

### TECHNICAL FIELD

The subject matter disclosed herein is generally directed to a platform for generating protein binding proteins with specificity for peptide-MHC (pMHC) complexes.

### BACKGROUND

The molecules that transport and present peptides on the cell surface are referred to as proteins of the major histocompatibility complex (MHC). The human leukocyte antigen (HLA) system is a gene complex encoding the major histocompatibility complex (MHC) proteins in humans. Human leukocyte antigen (HLA) class I glycoproteins (HLA-A, -B, and -C) are expressed on the surface of almost all nucleated cells in the human body and are required for presentation of short peptides for detection by T cell receptors. The HLA genes are the most polymorphic genes across the human population; with more than 16,200 distinct HLA class I allele variants identified as of May 2019 (Lefranc, M.-P. et al. IMGT®, the international ImMunoGeneTics information system® 25 years on. Nucleic Acids Res. 43, D413-22 (2015); and Robinson, J. et al. The IPD and IMGT/HLA database: allele variant databases. Nucleic Acids Res. 43, D423-31 (2015)). Each HLA allele is estimated to bind and present ˜1,000-10,000 unique peptides to T cells (Hunt DF, Henderson RA, Shabanowitz J, Sakaguchi K, Michel H, Sevilir N, Cox AL, Appella E, Engelhard VH. Characterization of peptides bound to the class I MHC molecule HLA-A2.1 by mass spectrometry. Science. 1992;255:1261-1263; Rammensee HG, Friede T, Stevanovic S. MHC ligands and peptide motifs: first listing. Immunogenetics. 1995;41:178-228; Vita R, Overton JA, Greenbaum JA, Ponomarenko J, Clark JD, Cantrell JR, Wheeler DK, Gabbard JL, Hix D, Sette A, Peters B. The immune epitope database (IEDB) 3.0. Nucleic Acids Res. 2015;43:D405-D412), less than 0.1% of the estimated 10 million potential 9-mer peptides from human protein-coding genes.

Cancer immunotherapies that redirect immune cells against cancer-specific antigens using chimeric antigen receptors (CARs) and bispecific immune cell engagers have revolutionized the treatment of hematologic malignancies and shown promise in solid tumors, but restrictions in antibody targeting to antigens to proteins presented on the cell surface have limited the application of these therapeutics. In contrast, TCR-based therapies are directed against antigens displayed as peptide fragments on the cell surface via the pMHC complex irrespective of the subcellular localization of the antigenic protein, vastly increasing the number of targetable antigens. TCR-mimetic binding proteins, which bind to peptide antigens displayed in the context of pMHC similar to natural TCRs, are a promising alternative for both CARs and bispecific immune cell engagers.

Recent advances in antibody library design and construction, in vitro display and selection methods, post-selection binder identification and maturation have helped increase the utility of in vitro antibody generation (Dübel, S., Stoevesandt, O., Taussig, M. J. & Hust, M. Generating recombinant antibodies to the complete human proteome. Trends Biotechnol. 28, 333-339 (2010)). For example, recently developed antibody library designs have been successfully used with in vitro display methods for engineering antibodies (McMahon, C. et al. Yeast surface display platform for rapid discovery of conformationally selective nanobodies. Nat. Struct. Mol. Biol. 25, 289-296 (2018); Moutel, S. et al. NaLi-H1: A universal synthetic library of humanized nanobodies providing highly functional antibodies and intrabodies. Elife 5, 1-31 (2016); Zimmermann, I. et al. Synthetic single domain antibodies for the conformational trapping of membrane proteins. Elife 7, 1-32 (2018)).

Heavy-chain-only antibodies bind to antigens solely based on the variable domain of their heavy chain, the VHH domain (also known as nanobodies). Nanobodies are increasingly used as functional antibody domains because of their small size (˜14 kD) (Muyldermans, S. Nanobodies: Natural single-domain antibodies. Annu. Rev. Biochem. 82, 775-797 (2013)) and high stability (Tₘ up to 90° C.) (Turner, K. B., Zabetakis, D., Goldman, E. R. & Anderson, G. P. Enhanced stabilization of a stable single domain antibody for SEB toxin by random mutagenesis and stringent selection. Protein Eng. Des. Sel. 27, 89-95 (2014)). Nanobody libraries have been successfully screened for binders by phage and yeast display (McMahon, C. et al. 2018; Huo, J. et al. Neutralizing nanobodies bind SARS-COV-2 spike RBD and block interaction with ACE2. Nat. Struct. Mol. Biol. (2020); Boder, E. T. & Wittrup, K. D. Yeast surface display for screening combinatorial polypeptide libraries. Nat. Biotechnol. 15, 553-557 (1997)). Conversely, cell-free approaches, such as ribosome display (Hanes, J. & Plückthun, A. In vitro selection and evolution of functional proteins by using ribosome display. Proc. Natl. Acad. Sci. U. S. A. 94, 4937-4942 (1997), are not limited by cell transformation and culture constraints.

Citation or identification of any document in this application is not an admission that such a document is available as prior art to the present invention.

### SUMMARY

In one aspect, the present invention provides for a method of generating binding proteins with specificity for a target peptide-MHC (pMHC) complex comprising: (a) performing ribosome display using a ribosome display library that generates a set of ribonucleoprotein complexes, each ribonucleoprotein complex comprising a candidate binding polypeptide and RNA encoding the candidate binding polypeptide; (b) negatively selecting ribonucleoprotein complexes displaying off-target binding polypeptides via binding the with one or more of a control pMHC, control peptide, and unloaded MHC immobilized on one or more solid supports; c. positively selecting ribonucleoprotein complexes displaying on-target candidate binding polypeptides via binding with a target pMHC immobilized on one or more solid supports; d. recovering RNAs encoding the positively selected on-target candidate binding polypeptides; e. optionally, repeating steps (a) to (d) based on the recovered RNAs in step (d) as the input for a new ribosome display library in step (a); and (f) sequencing the recovered RNAs to identify a final set of on-target binding polypeptides. In certain embodiments, the binding proteins encoded for in the ribosome display library are selected from the group consisting of a nanobody (VHH), antibody fragment (Fab), single-chain variable fragment (scFv), and non-antibody scaffold.

In certain embodiments, the method further comprises clustering the binding protein sequences containing similar binding domains. In certain embodiments, the binding proteins are clustered based on similarity of complementary determining regions (CDRs). In certain embodiments, the binding proteins clustered contain one or more of the same CDR. In certain embodiments, binding proteins are selected from one or more of the clusters having the largest number of members.

In certain embodiments, steps (a) to (d) are repeated more than 1, 2, 3, 4, 5, 6, 7, 8, 9,or 10 rounds. In certain embodiments, the method further comprises mutagenizing the binding proteins before repeating steps (a) to (d). In certain embodiments, CDRs are randomized.

In certain embodiments, the MHC molecule is a class I MHC or class II MHC. In certain embodiments, the MHC molecule is a human HLA allele.

In certain embodiments, the one or more solid supports are beads. In certain embodiments, the beads are magnetic beads.

In certain embodiments, the target peptide originates from a protein selected from the group consisting of tumor associated antigens, tumor specific antigens, neoantigens, self-antigens, allergens, or pathogen antigens. In certain embodiments, the neoantigens are derived from somatic mutations, RNA splicing, RNA editing, and/or neo-ORFs. In certain embodiments, the target pMHC is specific to a subject's MHC alleles and peptides capable of being presented by the subject's MHC alleles. In certain embodiments, the target pMHC is a pMHC isolated from a target cell. In certain embodiments, the target cell is obtained from a subject. In certain embodiments, the target cell is a cell line. In certain embodiments, the target cell is a tumor cell. In certain embodiments, the target cell is a cell targeted by an autoimmune response. In certain embodiments, the target cell is a cell line that is monoallelic for an MHC molecule. In certain embodiments, the target cell is an antigen presenting cell. In certain embodiments, the control peptide originates from a self-protein.

In certain embodiments, the method further comprises assembling a selected binding protein into a chimeric antigen receptor (CAR), bispecific engager molecule, diabody, triabody, tetrabody, or minibody.

In certain embodiments, the method further comprises validating the binding and activity of a selected binding protein. In certain embodiments, a binding protein representative of a cluster is validated. In certain embodiments, validating comprises expressing a recombinant binding protein and performing an ELISA against purified target pMHC. In certain embodiments, validating comprises expressing a recombinant binding protein and performing flow cytometry with beads coated with target pMHC or control pMHC. In certain embodiments, validating comprises expressing a recombinant binding protein and performing flow cytometry with cells displaying the target peptide or control peptide on expressed pMHC. In certain embodiments, the cells are TAP−/− cells. In certain embodiments, the recombinant binding protein is multimerized. In certain embodiments, the recombinant binding protein is biotinylated and tetramerized by the addition of streptavidin. In certain embodiments, the recombinant binding protein is fused to a multimerizing peptide sequence and self-assembled multimers are produced by in vitro transcription/translation (IVTT). In certain embodiments, validating comprises expressing a recombinant binding protein as a BITE or CAR T cell and performing a T cell cytotoxicity assay.

In another aspect, the present invention provides for a method of characterizing a peptide-MHC targeting binding protein comprising any method of validating the binding and activity of binding proteins according to any embodiment herein.

In another aspect, the present invention provides for a cell library comprising a population of cells expressing a plurality of pMHCs, each cell expressing a recombinant vector encoding a peptide, wherein each cell expresses a pMHC complex loaded with a peptide encoded for by the vector. In certain embodiments, each vector encodes for a peptide fused to an endoplasmic reticulum (ER) targeting signal peptide. In certain embodiments, each vector encodes for a single-chain pMHC comprising a peptide, beta-2-microglobulin (B2M), and an HLA allele connected by flexible peptide linkers. In certain embodiments, the cells in the library are TAP−/− cells.

In another aspect, the present invention provides for a method of characterizing a binding protein comprising incubating the binding protein with a cell library according to any embodiment herein; isolating bound cells; and quantifying the peptides encoded for by the vectors in the isolated cells.

These and other aspects, objects, features, and advantages of the example embodiments will become apparent to those having ordinary skill in the art upon consideration of the following detailed description of example embodiments.

### BRIEF DESCRIPTION OF THE DRAWINGS

An understanding of the features and advantages of the present invention will be obtained by reference to the following detailed description that sets forth illustrative embodiments, in which the principles of the invention may be utilized, and the accompanying drawings of which:

FIG. 1—Ribosome display process to select pMHC targeting proteins. (top) The workflow takes linear DNA library as input. Ribosome display links genotype (RNAs transcribed from DNA input library that are stop codon free, and stall ribosome at the end of the transcript) and phenotype (folded VHH protein tethered to ribosomes due to the lack of stop codon in the RNA). (bottom) Negative and positive selection cycles using pMHC for control peptides, unloaded MHC, and pMHC for a target peptide enriches DNA encoding for VHHs that binds immobilized pMHC targets. Graph comparing cluster size after consecutive cycles of ribosome display.

FIG. 2—ELISA data for selected nanobodies targeting P53 R175H.

FIG. 3—ELISA data for P53 R175H targeting nanobody binding affinity.

FIG. 4—Binding to peptide-pulsed T2 cells by nanobody tetramers.

FIG. 5—IVTT hexamer binding to pMHC coated beads.

FIG. 6—Characterization of on and off-target binding of nanobodies using IVTT and pMHC beads.

FIG. 7—IVTT hexamer binding to peptide-pulsed T2 cells.

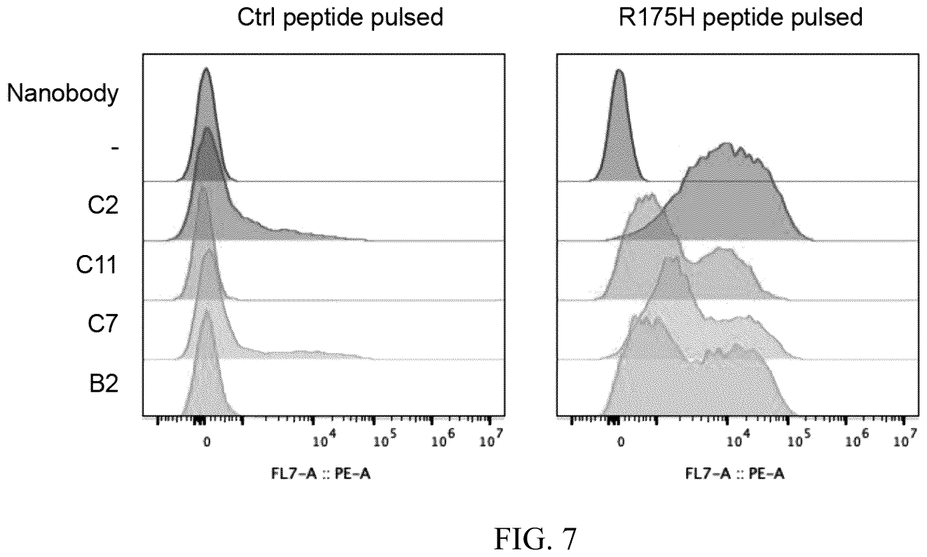

FIG. 8—Anti-CD3 nanobody BITE activation by IFN-γ ELISA. Targets are peptide pulsed T2 cells and ER-expressed peptides in T2 cells, and KMS cells.

The figures herein are for illustrative purposes only and are not necessarily drawn to scale.

### DETAILED DESCRIPTION OF THE EXAMPLE EMBODIMENTS

### General Definitions

Unless defined otherwise, technical and scientific terms used herein have the same meaning as commonly understood by one of ordinary skill in the art to which this disclosure pertains. Definitions of common terms and techniques in molecular biology may be found in Molecular Cloning: A Laboratory Manual, 2^{nd} edition (1989) (Sambrook, Fritsch, and Maniatis); Molecular Cloning: A Laboratory Manual, 4^{th} edition (2012) (Green and Sambrook); Current Protocols in Molecular Biology (1987) (F.M. Ausubel et al. eds.); the series Methods in Enzymology (Academic Press, Inc.): PCR 2: A Practical Approach (1995) (M. J. MacPherson, B. D. Hames, and G. R. Taylor eds.): Antibodies, A Laboratory Manual (1988) (Harlow and Lane, eds.): Antibodies A Laboratory Manual, 2^{nd} edition 2013 (E. A. Greenfield ed.); Animal Cell Culture (1987) (R.I. Freshney, ed.); Benjamin Lewin, Genes IX, published by Jones and Bartlet, 2008 (ISBN 0763752223); Kendrew et al. (eds.), The Encyclopedia of Molecular Biology, published by Blackwell Science Ltd., 1994 (ISBN 0632021829); Robert A. Meyers (ed.), Molecular Biology and Biotechnology: a Comprehensive Desk Reference, published by VCH Publishers, Inc., 1995 (ISBN 9780471185710); Singleton et al., Dictionary of Microbiology and Molecular Biology 2nd ed., J. Wiley & Sons (New York, N.Y. 1994), March, Advanced Organic Chemistry Reactions, Mechanisms and Structure 4th ed., John Wiley & Sons (New York, N.Y. 1992); and Marten H. Hofker and Jan van Deursen, Transgenic Mouse Methods and Protocols, 2^{nd} edition (2011).

As used herein, the singular forms “a”, “an”, and “the” include both singular and plural referents unless the context clearly dictates otherwise.

The term “optional” or “optionally” means that the subsequent described event, circumstance or substituent may or may not occur, and that the description includes instances where the event or circumstance occurs and instances where it does not.

The recitation of numerical ranges by endpoints includes all numbers and fractions subsumed within the respective ranges, as well as the recited endpoints.

The terms “about” or “approximately” as used herein when referring to a measurable value such as a parameter, an amount, a temporal duration, and the like, are meant to encompass variations of and from the specified value, such as variations of +/31 10% or less, +/−5% or less, +/−1% or less, and +/−0.1% or less of and from the specified value, insofar such variations are appropriate to perform in the disclosed invention. It is to be understood that the value to which the modifier “about” or “approximately” refers is itself also specifically, and preferably, disclosed.

As used herein, a “biological sample” may contain whole cells and/or live cells and/or cell debris. The biological sample may contain (or be derived from) a “bodily fluid”. The present invention encompasses embodiments wherein the bodily fluid is selected from amniotic fluid, aqueous humour, vitreous humour, bile, blood serum, breast milk, cerebrospinal fluid, cerumen (earwax), chyle, chyme, endolymph, perilymph, exudates, feces, female ejaculate, gastric acid, gastric juice, lymph, mucus (including nasal drainage and phlegm), pericardial fluid, peritoneal fluid, pleural fluid, pus, rheum, saliva, sebum (skin oil), semen, sputum, synovial fluid, sweat, tears, urine, vaginal secretion, vomit and mixtures of one or more thereof. Biological samples include cell cultures, bodily fluids, cell cultures from bodily fluids. Bodily fluids may be obtained from a mammal organism, for example by puncture, or other collecting or sampling procedures.

The terms “subject,” “individual,” and “patient” are used interchangeably herein to refer to a vertebrate, preferably a mammal, more preferably a human. Mammals include, but are not limited to, murines, simians, humans, farm animals, sport animals, and pets. Tissues, cells and their progeny of a biological entity obtained in vivo or cultured in vitro are also encompassed.

Various embodiments are described hereinafter. It should be noted that the specific embodiments are not intended as an exhaustive description or as a limitation to the broader aspects discussed herein. One aspect described in conjunction with a particular embodiment is not necessarily limited to that embodiment and can be practiced with any other embodiment(s). Reference throughout this specification to “one embodiment”, “an embodiment,” “an example embodiment,” means that a particular feature, structure or characteristic described in connection with the embodiment is included in at least one embodiment of the present invention. Thus, appearances of the phrases “in one embodiment,” “in an embodiment,” or “an example embodiment” in various places throughout this specification are not necessarily all referring to the same embodiment but may. Furthermore, the particular features, structures or characteristics may be combined in any suitable manner, as would be apparent to a person skilled in the art from this disclosure, in one or more embodiments. Furthermore, while some embodiments described herein include some but not other features included in other embodiments, combinations of features of different embodiments are meant to be within the scope of the invention. For example, in the appended claims, any of the claimed embodiments can be used in any combination.

Reference is made to International Patent Application publication WO2022067020A1. All publications, published patent documents, and patent applications cited herein are hereby incorporated by reference to the same extent as though each individual publication, published patent document, or patent application was specifically and individually indicated as being incorporated by reference.

### Overview

Applicants have developed methods for the generation and characterization of antibody and non-antibody binding proteins that bind specifically to a peptide presented by class I or class II MHC complexes (pMHC-targeting proteins), wherein the discovered pMHC-targeting proteins can be combined with other protein modules to construct immune effector molecules such as bispecific engagers and chimeric antigen receptors to construct therapeutics for immunooncology and autoimmunity. As used herein “pMHC-targeting proteins” refer to any protein capable of binding specifically to a peptide MHC complex. Since T cells bind to peptide bound MHC complexes through a T cell receptor (TCR) the pMHC-targeting proteins are also referred to as TCR-mimetic binding proteins because they mimic TCR binding to pMHC. The methods utilize a cell-free ribosome display platform for rapid, in vitro selection of binders against purified pMHC complexes and subsequent identification of their primary sequence using next-generation sequencing. Applicants have further developed a process for profiling the on-target and off-target binding properties of candidate proteins using in vitro transcription/translation of candidate proteins followed by assays against in vitro purified pMHC targets or peptides presented on the surface of pMHC expressing cells. In one aspect, the present invention provides for methods for discovering antibody and non-antibody proteins with specificity for peptide-MHC (pMHC) complexes using ribosome display. In another aspect, the present invention provides for methods for characterizing the binding and activity of pMHC-targeting proteins utilizing in vitro transcription/translation (IVTT). In another aspect, the present invention provides for methods for high-throughput profiling of the binding and activity pMHC-targeting proteins using cellular peptide-display libraries. In another aspect, the present invention provides for cellular peptide-display libraries. In another aspect, the present invention provides for pMHC binding proteins assembled into bispecific engager molecules or chimeric antigen receptors to activate/inhibit immune cells.

The methods are useful for discovery and profiling a large numbers of TCR-mimetic binding proteins useful for therapeutic applications in immunoncology, such as CAR-T and BITEs. The cancer antigens that are targetable by proteins designed using the disclosed methods include tumor-associated antigens and neoantigens derived from somatic mutations, RNA splicing, RNA editing, and neo-ORFs. In addition, the methods can develop binders against infectious disease targets displayed via pMHC, such as targets derived from HIV. The methods have additional applications in autoimmunity and transplantation, where selection of TCR-mimetic binders targeting self-antigens presented via class II MHC can be used to develop CAR-Treg cells. The methods can also be used to discover TCR-mimetic binders against pMHC targets that are difficult for traditional TCR-based therapeutics, such as HLA-E presented peptide.

### Platforms for Generating Peptide-Mhc Binding Proteins

In example embodiments, binding proteins having specificity to peptides bound to MHC (pMHC) proteins are generated. As used herein “specificity” refers to specific binding of the binding protein to the peptide bound to an MHC molecule. “Specific binding” of an antibody means that the binding protein exhibits appreciable affinity for a particular antigen or epitope and, generally, does not exhibit significant cross reactivity. “Appreciable” binding includes binding with an affinity of at least 25 μM. Binding proteins with affinities greater than 1×10⁷ M⁻¹ (or a dissociation coefficient of 1 μM or less or a dissociation coefficient of 1 nm or less) typically bind with correspondingly greater specificity. Values intermediate of those set forth herein are also intended to be within the scope of the present invention and binding proteins of the invention bind with a range of affinities, for example, 100 nM or less, 75 nM or less, 50 nM or less, 25 nM or less, for example 10 nM or less, 5 nM or less, 1 nM or less, or in embodiments 500 pM or less, 100 pM or less, 50 pM or less or 25 pM or less. A binding protein that “does not exhibit significant cross reactivity” is one that will not appreciably bind to an entity other than its target (e.g., a different epitope or a different molecule). For example, a binding protein that specifically binds to a target molecule will appreciably bind the target molecule but will not significantly react with non-target molecules or peptides. A binding protein specific for a particular epitope will, for example, not significantly cross react with remote epitopes on the same protein or peptide. Specific binding can be determined according to any art-recognized means for determining such binding. Preferably, specific binding is determined according to Scatchard analysis and/or competitive binding assays. As used herein, the term “affinity” refers to the strength of the binding of a single antigen-combining site with an antigenic determinant. Affinity depends on the closeness of stereochemical fit between binding protein combining sites and antigen determinants, on the size of the area of contact between them, on the distribution of charged and hydrophobic groups, etc. Binding protein affinity can be measured by equilibrium dialysis or by the kinetic BIACORE™ method. The dissociation constant, Kd, and the association constant, Ka, are quantitative measures of affinity.

Binding proteins

As used herein binding proteins refer to any protein capable of specifically binding an antigen or epitope of interest. Binding proteins can include antibodies, nanobodies (VHH), antibody fragments (Fab), single-chain variable fragments (scFv), and non-antibody scaffolds. Binding proteins targeting a pMHC can be selected from a library of binding proteins. As used herein “library” refers to a collection of binding proteins. In example embodiments, new libraries are formed during cycles of selection and/or mutagenesis. Libraries of binding proteins can include nucleic acids encoding for each member of the library (e.g., DNA, RNA).

### Antibodies

In certain embodiments, the present invention provides antibodies, antibody fragments, binding fragments of an antibody, or antigen binding fragments capable of binding to an antigen of interest (e.g., pMHC). The term “antibody” is used interchangeably with the term “immunoglobulin” herein, and includes intact antibodies, fragments of antibodies, e.g., Fab, F(ab′)2 fragments, and intact antibodies and fragments that have been mutated either in their constant and/or variable region (e.g., mutations to produce chimeric, partially humanized, or fully humanized antibodies, as well as to produce antibodies with a desired trait, e.g., enhanced binding and/or reduced FcR binding). The term “fragment” refers to a part or portion of an antibody or antibody chain comprising fewer amino acid residues than an intact or complete antibody or antibody chain. Fragments can be obtained via chemical or enzymatic treatment of an intact or complete antibody or antibody chain. Fragments can also be obtained by recombinant means. Exemplary fragments include Fab, Fab′, F(ab′)2, Fabc, Fd, dAb, VHH and scFv and/or Fv fragments.

The antibodies include “complementarity determining regions” or “CDRs” interspersed among “frame regions” or “FRs”, as defined herein. As used herein CDRs refer to variable regions in an antibody that provide for antigen specificity. In certain embodiments, specific CDRs identified can be used in a binding protein described further herein. In certain embodiments, one, two, or all three CDRs are used in a binding protein. In certain embodiments, CDR1 and CDR3 are used in a binding protein. In certain embodiments, CDR3 is used in a binding protein. In preferred embodiments, all three CDRs are used in a binding protein. As used herein, binding protein can refer to an entire antibody VHH domain or antibody as described herein. In example embodiments, frame region (FR) refers to the non-CDR regions or constant regions in the antibody. The frame regions in the antibodies of the present invention can be referred to as frame1, frame2, frame3 and frame4 (e.g., frame regions of a VHH).

The term “binding portion” of an antibody (or “antibody portion”) includes one or more complete domains, e.g., a pair of complete domains, as well as fragments of an antibody that retain the ability to specifically bind to a target molecule. It has been shown that the binding function of an antibody can be performed by fragments of a full-length antibody. Binding fragments are produced by recombinant DNA techniques, or by enzymatic or chemical cleavage of intact immunoglobulins. Binding fragments include Fab, Fab′, F(ab′)2, Fabc, Fd, dAb, Fv, single chains, VHH, single-chain antibodies, e.g., scFv, and single domain antibodies.

The term “antibody fragment” or “antigen-binding fragment” refers to a polypeptide fragment of an immunoglobulin or antibody that binds antigen or competes with intact antibody (i.e., with the intact antibody from which they were derived) for antigen binding (i.e., specific binding). As such these antibodies or fragments thereof are included in the scope of the invention, provided that the antibody or fragment binds specifically to a target molecule. Examples of portions of antibodies or epitope-binding proteins encompassed by the present definition include: (i) the Fab fragment, having VL, CL, VH and CH1 domains; (ii) the Fab′ fragment, which is a Fab fragment having one or more cysteine residues at the C-terminus of the CH1 domain; (iii) the Fd fragment having VH and CH1 domains; (iv) the Fd′ fragment having VH and CH1 domains and one or more cysteine residues at the C-terminus of the CHI domain; (v) the Fv fragment having the VL and VH domains of a single arm of an antibody; (vi) the dAb fragment (Ward et al., 341 Nature 544 (1989)) which consists of a VH domain or a VL domain that binds antigen; (vii) isolated CDR regions or isolated CDR regions presented in a functional framework; (viii) F(ab′)2 fragments which are bivalent fragments including two Fab′ fragments linked by a disulphide bridge at the hinge region; (ix) single chain antibody molecules (e.g., single chain Fv; scFv) (Bird et al., 242 Science 423 (1988); and Huston et al., 85 PNAS 5879 (1988)); (x) “diabodies” with two antigen binding sites, comprising a heavy chain variable domain (VH) connected to a light chain variable domain (VL) in the same polypeptide chain (see, e.g., EP 404,097; WO 93/11161; Hollinger et al., 90 PNAS 6444 (1993)); (xi) “linear antibodies” comprising a pair of tandem Fd segments (VH-Ch1-VH-Ch1) which, together with complementary light chain polypeptides, form a pair of antigen binding regions (Zapata et al., Protein Eng. 8 (10): 1057-62 (1995); and U.S. Pat. No. 5,641,870).

In example embodiments, the antibodies of the present invention are heavy chain antibodies. As used herein “heavy chain antibody,” “VHH” or “single-domain antibodies” (sdAbs) refers to an antibody which consists only of two heavy chains and lacks the two light chains usually found in antibodies (see, e.g., Henry and Mackenzie, Antigen recognition by single-domain antibodies: structural latitudes and constraints. MAbs. 2018 August-Sep; 10(6): 815-826). VHH can refer to an antibody or VHH domain. Single-domain antibodies (sdAb) are also known as a nanobody; an antibody fragment consisting of a single monomeric variable antibody domain. As used herein “VHH” is used interchangeably with “nanobody.” The ˜12-15 kDa variable domains of these antibodies (VHHs and VNARs) can be produced recombinantly and can recognize antigen in the absence of the remainder of the antibody heavy chain. In common antibodies, the antigen binding region consists of the variable domains of the heavy and light chains (VH and VL). Heavy-chain antibodies can bind antigens despite having only VH domains. In example embodiments, the heavy chain antibody is an antibody derived from cartilaginous fishes (immunoglobulin new antigen receptor (IgNAR)) or camelid ungulates. Non-limiting examples of camelids include dromedaries, camels, llamas, and alpacas.

In example embodiments, antibodies as described herein can be assembled into a diabody, triabody, tetrabody, or minibody. A diabody is a dimer of scFv which consists of VH and VL domains connected by a short peptide linker. The linker is too short to form intrachain pairing of VH and VL domains. Instead, two such scFv fragments are co-expressed to form multimers by inter-chain pairing (cross-over pairing) of VH and VL domains. A tribody and tetrabody refer to scFv trimers and teramers (see, e.g., Asano R, Koyama N, Hagiwara Y, et al. Anti-EGFR scFv tetramer (tetrabody) with a stable monodisperse structure, strong anticancer effect, and a long in vivo half-life. FEBS Open Bio. 2016;6(6):594-602). A minibody is a class of scFv-derived molecules with the structure VL-VH-CH3 (see, e.g., Hu S, Shively L, Raubitschek A, et al. Minibody: A novel engineered anti-carcinoembryonic antigen antibody fragment (single-chain Fv-CH3) which exhibits rapid, high-level targeting of xenografts. Cancer Res. 1996;56(13):3055-3061). A minibody can be a bivalent fusion molecule with two scFvs fused to CH3. The scFv targeting antigen A can be fused to the N-terminus of one of the CH3 domains and the scFv targeting antigen B to the other CH3. It can be further stabilized by C-terminal disulfide bonds.

In certain embodiments, antibodies prepared according to the present invention are substantially free of non-antibody protein. As used herein, a preparation of antibody protein having less than about 50% of non-antibody protein (also referred to herein as a “contaminating protein”), or of chemical precursors, is considered to be “substantially free.” 40%, 30%, 20%, 10% and more preferably 5% (by dry weight), of non-antibody protein, or of chemical precursors is considered to be substantially free. When the antibody protein or biologically active portion thereof is recombinantly produced, it is also preferably substantially free of culture medium, i.e., culture medium represents less than about 30%, preferably less than about 20%, more preferably less than about 10%, and most preferably less than about 5% of the volume or mass of the protein preparation.

In example embodiments, the antibodies of the present invention are monoclonal antibodies. As used herein, the term “monoclonal antibody” refers to a single antibody produced by any means, such as recombinant DNA technology. As used herein, the term “monoclonal antibody” also refers to an antibody derived from a clonal population of antibody-producing cells (e.g., B lymphocytes or B cells) which is homogeneous in structure and antigen specificity. The term “polyclonal antibody” refers to a plurality of antibodies originating from different clonal populations of antibody-producing cells which are heterogeneous in their structure and epitope specificity, but which recognize a common antigen. Monoclonal and polyclonal antibodies may exist within bodily fluids, as crude preparations, or may be purified, as described herein.

In certain embodiments, “humanized” forms of non-human antibodies contain amino acid residues in frame regions that resemble human antibody frame regions. In certain embodiments, frame regions of camelid antibodies or heavy chain antibodies are modified. In certain embodiments, humanized residues can be found in any human IGHV gene. In certain embodiments, the humanized residues are located in frame 2 or frame 4. In preferred embodiments, the humanized residues are located in frame 2 position 4, frame 2 position 11, frame 2 position 12, frame 2 position 14, frame 4 position 8. Humanized frames can be based on well characterized VHHs (Kirchhofer et al., 2010; Turner et al., 2014). These frames share high homology with the human IGHV3-23 or IGHJ4, but can be altered further as described herein (e.g., Frames 2 and 4).

In certain embodiments, “humanized” forms of non-human antibodies are chimeric antibodies that contain minimal sequence derived from non-human immunoglobulin (e.g., camelid). For the most part, humanized antibodies are human immunoglobulins (recipient antibody) in which residues from a hypervariable region of the recipient are replaced by residues from a hypervariable region of a non-human species (donor antibody) such as mouse, rat, rabbit or nonhuman primate having the desired specificity, affinity, and capacity. In some instances, FR residues of the human immunoglobulin are replaced by corresponding non-human residues. Furthermore, humanized antibodies may comprise residues that are not found in the recipient antibody or in the donor antibody. These modifications are made to further refine antibody performance. In general, the humanized antibody will comprise substantially all of at least one, and typically two, variable domains, in which all or substantially all of the hypervariable regions correspond to those of a non-human immunoglobulin and all or substantially all of the FR regions are those of a human immunoglobulin sequence. The humanized antibody optionally also will comprise at least a portion of an immunoglobulin constant region (Fc), typically that of a human immunoglobulin.

In certain embodiments, the antibodies and CDRs of the present invention can be transferred to another antibody type (e.g., to chimeric antigen receptors, bi-specific antibodies, T cell receptors) to generate chimeric antibodies. It is intended that the term “antibody type” encompass any Ig class or any Ig subclass (e.g., the IgG1, IgG2, IgG3, and IgG4 subclasses of IgG) obtained from any source (e.g., humans and non-human primates, and in rodents, lagomorphs, caprines, bovines, equines, ovines, etc.).

The term “Ig class” or “immunoglobulin class”, as used herein, refers to the five classes of immunoglobulin that have been identified in humans and higher mammals, IgG, IgM, IgA, IgD, and IgE. The term “Ig subclass” refers to the two subclasses of IgM (H and L), three subclasses of IgA (IgA1, IgA2, and secretory IgA), and four subclasses of IgG (IgG1, IgG2, IgG3, and IgG4) that have been identified in humans and higher mammals. The antibodies can exist in monomeric or polymeric form; for example, IgM antibodies exist in pentameric form, and IgA antibodies exist in monomeric, dimeric, or multimeric form.

The term “IgG subclass” refers to the four subclasses of immunoglobulin class IgG-IgG1, IgG2, IgG3, and IgG4 that have been identified in humans and higher mammals by the heavy chains of the immunoglobulins, V1-γ4, respectively. The term “single-chain immunoglobulin” or “single-chain antibody” (used interchangeably herein) refers to a protein having a two-polypeptide chain structure consisting of a heavy and a light chain, said chains being stabilized, for example, by interchain peptide linkers, which has the ability to specifically bind antigen. The term “domain” refers to a globular region of a heavy or light chain polypeptide comprising peptide loops (e.g., comprising 3 to 4 peptide loops) stabilized, for example, by β pleated sheet and/or intrachain disulfide bond. Domains are further referred to herein as “constant” or “variable”, based on the relative lack of sequence variation within the domains of various class members in the case of a “constant” domain, or the significant variation within the domains of various class members in the case of a “variable” domain. Antibody or polypeptide “domains” are often referred to interchangeably in the art as antibody or polypeptide “regions”. The “constant” domains of an antibody light chain are referred to interchangeably as “light chain constant regions”, “light chain constant domains”, “CL” regions or “CL” domains. The “constant” domains of an antibody heavy chain are referred to interchangeably as “heavy chain constant regions”, “heavy chain constant domains”, “CH” regions or “CH” domains). The “variable” domains of an antibody light chain are referred to interchangeably as “light chain variable regions”, “light chain variable domains”, “VL” regions or “VL” domains). The “variable” domains of an antibody heavy chain are referred to interchangeably as “heavy chain constant regions”, “heavy chain constant domains”, “VH” regions or “VH” domains).

The term “region” can also refer to a part or portion of an antibody chain or antibody chain domain (e.g., a part or portion of a heavy or light chain or a part or portion of a constant or variable domain, as defined herein), as well as more discrete parts or portions of said chains or domains. For example, light and heavy chains or light and heavy chain variable domains include “complementarity determining regions” or “CDRs” interspersed among “frame regions” or “FRs”, as defined herein.

The term “conformation” refers to the tertiary structure of a protein or polypeptide (e.g., an antibody, antibody chain, domain, or region thereof). For example, the phrase “light (or heavy) chain conformation” refers to the tertiary structure of a light (or heavy) chain variable region, and the phrase “antibody conformation” or “antibody fragment conformation” refers to the tertiary structure of an antibody or fragment thereof.

### Non-Antibody Scaffolds

The terms “non-antibody scaffold,” “antibody-like protein scaffolds,” or “engineered protein scaffolds” broadly encompasses proteinaceous non-immunoglobulin specific-binding agents, typically obtained by combinatorial engineering (such as site-directed random mutagenesis in combination with phage display or other molecular selection techniques). Usually, such scaffolds are derived from robust and small soluble monomeric proteins (such as Kunitz inhibitors or lipocalins) or from a stably folded extra-membrane domain of a cell surface receptor (such as protein A, fibronectin or the ankyrin repeat).

Such scaffolds have been extensively reviewed in Binz et al. (Engineering novel binding proteins from nonimmunoglobulin domains. Nat Biotechnol 2005, 23:1257-1268), Gebauer and Skerra (Engineered protein scaffolds as next-generation antibody therapeutics. Curr Opin Chem Biol. 2009, 13:245-55), Gill and Damle (Biopharmaceutical drug discovery using novel protein scaffolds. Curr Opin Biotechnol 2006, 17:653-658), Skerra (Engineered protein scaffolds for molecular recognition. J Mol Recognit 2000, 13:167-187), and Skerra (Alternative non-antibody scaffolds for molecular recognition. Curr Opin Biotechnol 2007, 18:295-304), and include without limitation affibodies, based on the Z-domain of staphylococcal protein A, a three-helix bundle of 58 residues providing an interface on two of its alpha-helices (Nygren, Alternative binding proteins: Affibody binding proteins developed from a small three-helix bundle scaffold. FEBS J 2008, 275:2668-2676); engineered Kunitz domains based on a small (ca. 58 residues) and robust, disulphide-crosslinked serine protease inhibitor, typically of human origin (e.g., LACI-D1), which can be engineered for different protease specificities (Nixon and Wood, Engineered protein inhibitors of proteases. Curr Opin Drug Discov Dev 2006, 9:261-268); monobodies or adnectins based on the 10th extracellular domain of human fibronectin III (10Fn3), which adopts an Ig-like beta-sandwich fold (94 residues) with 2-3 exposed loops, but lacks the central disulphide bridge (Koide and Koide, Monobodies: antibody mimics based on the scaffold of the fibronectin type III domain. Methods Mol Biol 2007, 352:95-109); anticalins derived from the lipocalins, a diverse family of eight-stranded beta-barrel proteins (ca. 180 residues) that naturally form binding sites for small ligands by means of four structurally variable loops at the open end, which are abundant in humans, insects, and many other organisms (Skerra, Alternative binding proteins: Anticalins-harnessing the structural plasticity of the lipocalin ligand pocket to engineer novel binding activities. FEBS J 2008, 275:2677-2683); DARPins, designed ankyrin repeat domains (166 residues), which provide a rigid interface arising from typically three repeated beta-turns (Stumpp et al., DARPins: a new generation of protein therapeutics. Drug Discov Today 2008, 13:695-701); avimers (multimerized LDLR-A module) (Silverman et al., Multivalent avimer proteins evolved by exon shuffling of a family of human receptor domains. Nat Biotechnol 2005, 23:1556-1561); and cysteine-rich knottin peptides (Kolmar, Alternative binding proteins: biological activity and therapeutic potential of cystine-knot miniproteins. FEBS J 2008, 275:2684-2690).

### Immune System and Antigen Presentation

The present invention provides methods for generating binding proteins specific for peptides bound to HLA alleles. The immune system can be classified into two functional subsystems: the innate and the acquired immune system. The innate immune system is the first line of defense against infections, and most potential pathogens are rapidly neutralized by this system before they can cause, for example, a noticeable infection. The acquired immune system reacts to molecular structures, referred to as antigens, of the intruding organism or diseased cell (e.g., tumor cells). There are two types of acquired immune reactions, which include the humoral immune reaction and the cell-mediated immune reaction. In the humoral immune reaction, antibodies secreted by B cells into bodily fluids bind to antigens, leading to the elimination of the pathogen or diseased cell through a variety of mechanisms, e.g., complement-mediated lysis. In the cell-mediated immune reaction, T-cells capable of destroying other cells are activated. For example, if proteins associated with a disease are present in a cell, they are fragmented proteolytically to peptides within the cell. Specific cell proteins then attach themselves to the antigen or peptide formed in this manner and transport them to the surface of the cell, where they are presented to the molecular defense mechanisms, in particular T-cells, of the body. Cytotoxic T cells recognize these antigens and kill the cells that harbor the antigens.

The molecules that transport and present peptides on the cell surface are referred to as proteins of the major histocompatibility complex (MHC). MHC proteins are classified into two types, referred to as MHC class I and MHC class II. The structures of the proteins of the two MHC classes are very similar; however, they have very different functions. Proteins of MHC class I are present on the surface of almost all cells of the body, including most tumor cells. MHC class I proteins are loaded with antigens that usually originate from endogenous proteins or from pathogens present inside cells, and are then presented to naive or cytotoxic T-lymphocytes (CTLs). MHC class II proteins are present on dendritic cells, B-lymphocytes, macrophages, and other antigen-presenting cells. They mainly present peptides, which are processed from external antigen sources, i.e., outside of the cells, to T-helper (Th) cells. Most of the peptides bound by the MHC class I proteins originate from cytoplasmic proteins produced in the healthy host cells of an organism itself, and do not normally stimulate an immune reaction. Accordingly, cytotoxic T-lymphocytes that recognize such self-peptide-presenting MHC molecules of class I are deleted in the thymus (central tolerance) or, after their release from the thymus, are deleted or inactivated, i.e., tolerized (peripheral tolerance). MHC molecules are capable of stimulating an immune reaction when they present peptides to non-tolerized T-lymphocytes. Cytotoxic T-lymphocytes have both T-cell receptors (TCR) and CD8 molecules on their surface. T-Cell receptors are capable of recognizing and binding peptides complexed with the molecules of MHC class I. Each cytotoxic T-lymphocyte expresses a unique T-cell receptor which is capable of binding specific MHC/peptide complexes.

The peptide antigens attach themselves to the molecules of MHC class I by competitive affinity binding within the endoplasmic reticulum, before they are presented on the cell surface. Here, the affinity of an individual peptide antigen is directly linked to its amino acid sequence and the presence of specific binding motifs in defined positions within the amino acid sequence. If the sequence of such a peptide is known, it is possible to manipulate the immune system against diseased cells using, for example, peptide vaccines. The human leukocyte antigen (HLA) system is a gene complex encoding the major histocompatibility complex (MHC) proteins in humans.

By “proteins or molecules of the major histocompatibility complex (MHC)”, “MHC molecules”, “MHC proteins” or “HLA proteins” is thus meant proteins capable of binding peptides resulting from the proteolytic cleavage of protein antigens and representing potential T cell epitopes, transporting them to the cell surface and presenting them there to specific cells, in particular cytotoxic T-lymphocytes or T-helper cells.

MHC molecules of class I consist of a heavy chain and a light chain and are capable of binding a peptide of about 8 to 11 amino acids, but usually 9 or 10 amino acids, if this peptide has suitable binding motifs, and presenting it to cytotoxic T-lymphocytes. The peptide bound by the MHC molecules of class I originates from an endogenous protein antigen. The peptide antigens attach themselves to the molecules of MHC class I by competitive affinity binding within the endoplasmic reticulum before they are presented on the cell surface. The heavy chain of the MHC molecules of class I is preferably an HLA-A, HLA-B or HLA-C monomer (but also including HLA-G and HLA-E), and the light chain is β-2-microglobulin (B2M).

MHC molecules of class II consist of an α-chain and a β-chain and are capable of binding a peptide of about 15 to 24 amino acids if this peptide has suitable binding motifs, and presenting it to T-helper cells. The peptide bound by the MHC molecules of class II usually originates from an extracellular of exogenous protein antigen. The α-chain and the β-chain are in particular HLA-DR, HLA-DQ and HLA-DP monomers.

Subject specific HLA alleles or HLA genotype of a subject may be determined by any method known in the art. In example embodiments, genotyping is determined by sequencing, polymerase chain reaction, or hybridization. In example embodiments, the present invention includes whole genome sequencing. Whole genome sequencing (also known as WGS, full genome sequencing, complete genome sequencing, or entire genome sequencing) is the process of determining the complete DNA sequence of an organism's genome at a single time. In example embodiments, targeted sequencing is used in the present invention (see, e.g., Mantere et al., PLOS Genet 12 e1005816 2016; and Carneiro et al. BMC Genomics, 2012 13:375). In example embodiments, HLA genotypes are determined by any method described in International Patent Application number PCT/US2014/068746, published Jun. 11, 2015 as WO2015085147.

Selection of Target peptides

In example embodiments, the invention takes as an initial input a target peptide or a set of target peptides. In example embodiments, target peptides can be specific to a disease. For example, peptides presented on the surface of tumor cells or cells associated with autoimmunity. Target peptides can originate from tumor associated antigens, tumor specific antigens, neoantigens, self-antigens, allergens, or pathogen antigens. In example embodiments, the neoantigens are derived from somatic mutations, RNA splicing, RNA editing, and/or neo-ORFs (also known as, novel untranslated open reading frame (nuORF)). As used herein, the term “neoantigen” or “neoantigenic” means (1) a class of tumor antigens that arises from a tumor-specific mutation(s) which alters the amino acid sequence of genome encoded proteins; (2) a class of tumor antigens having tumor specific expression that arises from retained introns, alternative open reading frames (ORFs) within coding genes, antisense transcripts, defective ribosomal products (DRiPs), “non-coding” regions of the genome, 5′ and 3′ untranslated regions (UTRs), overlapping yet out-of-frame alternative ORFs in annotated protein-coding genes, long non-coding RNAs (IncRNAs), pseudogenes and other transcripts currently annotated as non-protein coding; or (3) novel unannotated open reading frames (nuORFs) that arise from a tumor-specific mutation(s) in unannotated open reading frames. The neoantigens, or neoepitopes, or neopolypeptides may also be subject specific. Methods of identifying neoantigens have been described, for example, by whole genome sequencing and identifying non-silent somatic mutations (see, e.g., Rajasagi et al., 2014, Systematic identification of personal tumor-specific neoantigens in chronic lymphocytic leukemia. Blood. 2014 Jul. 17;124(3):453-62; US patent application publication numbers US20220062394A1; US20190060428A1; US20160101170A1; US20160339090A1; US20180153975A1; and U.S. Pat. No. 10,426,824B1). As used herein “tumor associated antigen” or “TAA” refers to antigens that have elevated levels on tumor cells, but are also expressed at lower levels on healthy cells. As used herein “tumor specific antigen” refers to antigens that are found on cancer cells only, not on healthy cells. Self-antigens are by convention antigens in the body of an individual. In regards to autoimmune diseases, self-antigens are those cellular proteins, peptides, enzyme complexes, ribonucleoprotein complexes, DNA, and post-translationally modified antigens against which autoantibodies are directed. Pathogen antigens can be any antigen derived from a virus, bacteria, or any pathogen that infects a subject.

In example embodiments, the target peptide or set of target peptides may be obtained from a subject or group of subjects in need of an immune response or modified immune response. In example embodiments, target peptides can be identified in a peptide sequence database (e.g., derived from sequencing of pMHC molecules isolated from subjects having a specific condition where an immunogenic composition would be useful). In example embodiments, target peptides can be identified by analyzing a genome sequencing database and predicting antigenic peptides that bind to specific MHC alleles (e.g., derived from whole genome sequencing of disease related cells from subjects having a specific condition where an immunogenic composition would be useful). In example embodiments, target peptides can be identified by analyzing a whole genome sequence from a subject in need thereof and predicting antigenic peptides that bind to specific MHC alleles for the subject (e.g., for personalized therapy).

Immunological therapies (e.g., cancer, infections, autoimmune diseases) rely on accurate selection of peptides to target (e.g., tumor-specific neoepitopes or viral epitopes). Given the patient's particular complement of HLA alleles, the ability to predict which epitopes will be presented is a fundamental prerequisite for successful immunological therapeutic design. Additionally, given the unique accumulation of mutations in different tumors as well as the patient's particular complement of HLA alleles, the ability to predict which epitopes will be presented is a fundamental prerequisite for successful cancer targeting design. Additionally, epitopes recognized in autoimmune diseases for patients having specific HLA alleles is a prerequisite for designing treatments aimed at blocking an immune response against the target proteins (e.g., by targeting CAR Tregs).

In example embodiments, target peptides are identified by isolating MHC complexes from a subject and profiling peptides presented on their HLA molecules via mass spectrometry. In example embodiments, peptides are identified on tumor cells obtained from the subject. Advances in mass spectrometry (MS)-based immunopeptidomics approaches have enabled identification of endogenously processed and presented HLA peptides (Garde et al. 2019 Immunogenetics 71 (7): 445-54; Chong et al. 2018 Molecular & Cellular Proteomics: MCP 17 (3): 533-48; Klaeger et al. 2021 Molecular & Cellular Proteomics: MCP 20 (August): 100133; Vizcaíno et al. 2020 Molecular & Cellular Proteomics: MCP 19 (1): 31-49; Andreatta et al. 2019 Proteomics 19 (4): e1800357; Marcu et al. 2021 Journal for Immunotherapy of Cancer 9 (4); van Balen et al. 2020 Journal of Immunology 204 (12): 3273-82; Abelin et al. 2019 Immunity 51 (4): 766-79.e17).

Any method of predicting peptides that bind to a specific HLA allele can be used to select target peptides to be used in generating pMHC binding proteins. Rules for peptide binding to HLA molecules have been studied extensively for a subset of HLA alleles (Vita R, Overton JA, Greenbaum J A, Ponomarenko J, Clark J D, Cantrell J R, Wheeler D K, Gabbard J L, Hix D, Sette A, Peters B. The immune epitope database (IEDB) 3.0. Nucleic Acids Res. 2015;43: D405-D412) and have been encoded in modern advanced neural-network-based algorithms (Hoof I, Peters B, Sidney J, Pedersen L E, Sette A, Lund O, Buus S, Nielsen M. NetMHCpan, a method for MHC class I binding prediction beyond humans. Immunogenetics. 2009;61:1-13; Lundegaard C, Lamberth K, Harndahl M, Buus S, Lund O, Nielsen M. NetMHC-3.0: accurate web accessible predictions of human, mouse and monkey MHC class I affinities for peptides of length 8-11. Nucleic Acids Res. 2008;36: W509-12; and Reynisson, Birkir, Bruno Alvarez, Sinu Paul, Bjoern Peters, and Morten Nielsen. 2020. “NetMHCpan-4.1 and NetMHCIIpan-4.0: Improved Predictions of MHC Antigen Presentation by Concurrent Motif Deconvolution and Integration of MS MHC Eluted Ligand Data.” Nucleic Acids Research 48 (W1): W449-54).

In example embodiments, candidate target peptides are identified by analyzing the HLA genotype and sequences of candidate peptides identified by whole genome sequencing of a subject in need thereof with a machine which has been trained with a peptide sequence database obtained by mass spectrometry sequencing of peptides bound to HLA I alleles in monoallelic cell lines (see, e.g., US20190346442A1; and US20210382068A1). In example embodiments, peptides are identified by (a) nucleic acid sequencing of a sample of the subject's tumor and a non-tumor sample of the subject; (b) determining based on the nucleic acid sequencing non-silent mutations present in the genome of cancer cells of the subject but not in normal tissue from the subject; and (c) selecting from the identified non-silent mutations one or more subject-specific peptides, each having a different tumor neo-epitope that is an epitope specific to the tumor of the subject and each having a predictive score indicative of binding an HLA protein of the subject, wherein said predictive score is determined by analyzing the sequence of peptides derived from the non-silent mutations. In example embodiments, peptides capable of binding to an HLA allele are predicted by: identifying structural features indicative of occupancy of one or more candidate peptides on the binding pocket of the HLA allele; and predicting the candidate peptides that bind to the HLA molecule using a machine learning algorithm model trained using structural features extracted from one or more output models simulating occupancy of one or more binding peptides and one or more non-binding peptides on the HLA binding pocket in a crystal structure of the HLA allele or the crystal structure of a similar HLA allele (see, e.g., US20210104294A1).

In example embodiments, candidate target peptides are identified by analyzing an annotated genome sequence of a target pathogen, a commensal microorganism, or a diseased cell thereof with a machine which has been trained with a peptide sequence database obtained by mass spectrometry sequencing of mature peptide-MHCII complexes immunoprecipitated from primary bone marrow-derived dendritic cells (BMDCs) (see, e.g., US20220293215A1; Taylor H B, Klaeger S, Clauser K R, et al. MS-Based HLA-II Peptidomics Combined With Multiomics Will Aid the Development of Future Immunotherapies. Mol Cell Proteomics. 2021;20:100116; and Graham DB, Luo C, O'Connell DJ, et al. Antigen discovery and specification of immunodominance hierarchies for MHCII-restricted epitopes. Nat Med. 2018;24(11):1762-1772).

### Platform for Enriching PMHC Binding Proteins

In one aspect, the present invention provides a platform for generating binding proteins specific to pMHC complexes. The platform includes ribosome display and includes both negative and positive selection steps that allow for enrichment of binding proteins specific to a target pMHC. The platform provides for identifying families of binding proteins capable of binding to a target pMHC of interest. The platform provides for affinity maturation by mutating selected binding proteins. The platform also provides for validation of binding proteins.

### Ribosome Display

The present invention provides methods that allow ribosome display to be used to select protein binding proteins that can discriminate small peptides bound to specific MHC molecules using a combination of unique selection steps. As used herein ribosome display refers to an in vitro selection and evolution technology for proteins and peptides from large libraries (see, e.g., Zahnd, et al., Ribosome display: selecting and evolving proteins in vitro that specifically bind to a target. Nat Methods 4, 269-279 (2007)). Ribosome display also refers to a technique used to perform in vitro protein evolution to create proteins that can bind to a desired ligand. The process results in translated proteins that are associated with their mRNA progenitor (ribonucleoprotein complexes) which is used, as a complex, to bind to an immobilized ligand in a selection step. The mRNA present in ribonucleoprotein complexes that bind well are then reverse transcribed to cDNA and their sequence identified.

In example embodiments, the platform utilizes a binding protein library configured for use in ribosome display including nucleic acid sequences encoding for the binding proteins (ribosome display library). The binding proteins can be selected from any of the binding proteins discussed herein. In example embodiments, the platform utilizes VHH sequences. In preferred embodiments, the library is a DNA library, each DNA sequence in the library encoding for a binding protein operably linked to a promoter sequence for use in in vitro transcription/translation (IVTT). The promoter sequence is preferably compatible with in vitro transcription/translation systems (e.g., T7 promoter). In example embodiments, the nucleic acid sequences encoding for the binding proteins do not include a stop codon, whereby the mRNA and translated protein is not released by ribosomes. Thus, in example embodiments, the library is transcribed into mRNAs encoding for each binding protein. The mRNA can then be translated to generate the binding protein polypeptide. In example embodiments, library members are cloned into vectors including the promoter sequence. In an example embodiment, PCR primers specific to the vector are used to amplify out each library member to generate double stranded DNA for input to ribosome display.

Libraries can include a varying number of members, such as up to about 100 members, such as up to about 1,000 members, such as up to about 5,000 members, such as up to about 10,000 members, such as up to about 100,000 members, such as up to about 500,000 members, or even more than 500,000 members. In one example, the methods can involve providing a library containing a large number of potential binding proteins. Such libraries are then screened by the methods disclosed herein to identify those library members that display a desired characteristic activity (e.g., binding to target pMHC).

In example embodiments, a binding protein encoded for by the library includes CDR sequences that can be randomized (e.g., randomized at CDR1, CDR2, and/or CDR3). In example embodiments, the library includes binding proteins derived from an initial binding protein that differ in having randomized CDR sequences. In example embodiments, the library is generated by analyzing naturally occurring antibody frameworks (e.g., heavy chain antibodies). Templates are then generated with selected frameworks. In certain embodiments, CDR regions are chosen having the most variation between different antibody frameworks. Sets of primer pairs can be generated to randomly mutate each CDR sequence in each framework (see, e.g., International Patent Application publication WO2022067020A1).

In example embodiments, the antigen of interest (e.g., pMHC) and control peptides are immobilized to a solid support (i.e., surface-immobilized target), such as magnetic particles, latex beads, nanoparticles, macro-beads, membranes, microplates, flow cells, columns, array surfaces, dipsticks and a host of other devices that facilitate the capture of specific biomolecules. Any method of immobilization to a solid support can be used, for example, biotin labeled proteins binding to a streptavidin surface, or click chemistry (see, e.g., Kolb, H. C., Finn, M. G. and Sharpless, K. B. (2001), Click Chemistry: Diverse Chemical Function from a Few Good Reactions. Angewandte Chemie International Edition, 40:2004-2021; and Hoyle, Charles E. and Bowman, Christopher N. (2010), Thiol-Ene Click Chemistry. Angewandte Chemie International Edition, 49:1540-1573). Negative selection can include one or more of a control pMHC, control peptide, and unloaded MHC immobilized on a single solid support (e.g., a bead) or on separate solid supports. The solid supports are then used to select for translated binding proteins capable of binding the antigen of interest and not control peptides.

In example embodiments, the ribonucleoprotein complexes are negatively selected with control peptides. The unbound ribonucleoprotein complexes are then used for positive selection. In preferred embodiments, negative selection is performed before positive selection to remove binding proteins that non-specifically bind. Control peptides include a control pMHC, control peptide, and unloaded MHC. For example, a control peptide can be any non-target peptide that binds to the MHC molecule. For example, the control peptide can be a self-peptide. The control peptide can be a mutated target peptide. In separate rounds of ribosome display different control peptides may be used. In separate rounds of ribosome display, negative selection can use peptides derived from the target peptide that include decreasing numbers of mutations from the target peptide. The control pMHC can include the control peptide. Negative selection can be performed in a single step or separate steps (e.g., one step each for any combination of control peptides). In example embodiments, the ribonucleoprotein complexes are positively selected with the target pMHC (selection of target peptides is described further herein). In example embodiments, during positive selection the solid supports are washed to remove unbound ribonucleoprotein complexes. In example embodiments, the stringency of the washing and binding steps is adjusted to increase binding stringency. In example embodiments, stringency is increased by increasing the ionic strength of the buffers. In certain embodiments, stringency is increased by adding or increasing the concentration of detergent in the buffers. The binding and washing is preferably performed at about 4° C., however stringency can be changed by increasing the temperature. In example embodiments, binding time is adjusted. In example embodiments, binding time in initial cycles of ribosome display may be longer and in successive cycles decreased to increase stringency. For example, binding proteins that bind overnight can be identified in an early round. In example embodiments, binding is performed overnight (about 12 hours), 4 hours, 3, hours, 2 hours, 1 hour or less than 1 minute. In example embodiments, binding time is the same for negative and positive selection. In example embodiments, binding time is greater for negative selection as compared to positive selection. In example embodiments, binding is performed in buffers containing Mg²⁺ ions at concentrations of 5 mM or less.

After the solid support is washed mRNA can be isolated. The mRNA can be converted to cDNA by reverse transcription PCR (RT-PCR). In preferred embodiments, the PCR reaction in the RT-PCR is performed using a mixture of two DNA polymerases, wherein one type is a DNA polymerase without or having extremely weak strand displacement activity (e.g., Phusion® High-Fidelity DNA Polymerase, New England Biolabs) and the other type is a DNA polymerase with strong strand displacement activity (e.g., Deep Vent® DNA Polymerase, New England Biolabs). The cDNA can then be used as input for successive rounds of ribosome display. In preferred embodiments, 3 rounds are performed, however 1, 2, 3, 4, 5, 6, 7, 8, 9 or 10 or more rounds can be performed. In example embodiments, the number of rounds is saturated when the same library members are isolated at each round.

The platform also includes computational steps capable of clustering binding proteins having similar CDRs. In example embodiments, clustering allows for families of related binding proteins to be identified, such that one or a few representative binding proteins from each family can be further assayed or validated to determine binding activity of different families. In example embodiments, every family identified is further validated. In example embodiments, 1, 2, 3, 4, 5, 6, 7, 8, 9, 10 or 20 or more binding proteins clustered in a family are validated.

In example embodiments, binding proteins identified are further mutated by one or more rounds of affinity maturation. Affinity maturation refers to the introduction of random mutations across the full length of selected binding protein sequences (i.e., also including frame regions) and performing ribosome display with the antigen of interest. One method of affinity maturation is error-prone PCR of vectors comprising the library DNA sequences. In example embodiments, binding during ribosome display after affinity maturation is performed for 1 minute or less. In a preferred embodiment, incubation time of the binding step can be between 5 seconds to 1 minute to impose a stringent selection condition on affinity matured binding proteins. Beneficial mutations can be determined by determining the change of frequency of each observed amino acid at each position of the binding protein following affinity maturation.

### Validation of Binding Proteins

In example embodiments, the platform includes assays capable of validating pMHC binding of selected binding proteins and assaying activity when assembled into a therapeutic binding protein (described further herein).

In example embodiments, the validation assay is an immunoassay (e.g., ELISA, radioimmunoassay, fluorescent, chemiluminescence, fluorescence resonance energy transfer (FRET) or time resolved-FRET (TR-FRET) immunoassays, immunoprecipitation, immunocytochemistry, immunohistochemistry, or flow cytometry) (see, e.g., ImmunoAssay: A Practical Guide, edited by Brian Law, published by Taylor & Francis, Ltd., 2005 edition). These immunoassay formats have been designed to provide qualitative, semi-quantitative, and quantitative results. Quantitative results may be generated by determining the concentration of pMHC detected by binding protein. Quantitative results may be generated through the use of a standard curve created with known concentrations of the pMHC to be detected. In example embodiments, the binding protein is conjugated to a detectable label. Methods of detecting and/or quantifying a detectable label or signal generating material depend on the nature of the label. The labels can be, without limitation, fluorescent, luminescent, or radioactive or they may absorb visible or ultraviolet light. Examples of detectors suitable for detecting such detectable labels include, without limitation, x-ray film, radioactivity counters, scintillation counters, spectrophotometers, colorimeters, fluorometers, luminometers, and densitometers.

In one example embodiment, binding protein sequences are cloned into expression vectors and recombinant protein is purified (e.g., expressed and purified from E. coli). Purified binding proteins can be assayed via ELISA to determine binding to an uncoated plate, and pMHC loaded with target peptide or control peptides. In an example embodiment, peptides mutated at one or more residues are used. Binding proteins with specific binding to target pMHCs can be further assayed to determine binding affinity (EC50) using ELISA.

In another example embodiment, binding protein sequences are cloned into expression vectors, expressed and purified, biotinylated in vitro, and tetramerized by the addition of streptavidin. Targets displaying target or control peptide on expressed pMHC are produced by pulsing TAP−/− cells with peptide, expressing peptides fused to an ER signal sequence, or transducing cells with single-chain pMHC, and binding protein tetramer binding is analyzed with flow cytometry to determine on-target and off-target binding. In an example embodiment, peptides mutated at one or more residues are used. Single-chain pMHC can include (1) a target or control peptide sequence, (2) beta-2-microglobulin (B2M) sequence, and (3) an MHCI allele, all connected by flexible peptide linkers. In the MHC-I antigen processing pathway, peptides are generated from cytosolic proteins by proteasomes and then transported into the lumen of the endoplasmic reticulum via the TAP, an ATP-dependent peptide transporter that is composed of two subunits, TAPI and TAP2. Deficiency of either TAP1 or TAP2 blocks TAP function. TAP-deficient cells fail to transport cytosolic peptides into the endoplasmic reticulum, reducing the supply and repertoire of peptides available for binding to MHC-I (see, e.g., Johnsen A K, Templeton D J, Sy M, Harding C V. Deficiency of transporter for antigen presentation (TAP) in tumor cells allows evasion of immune surveillance and increases tumorigenesis. J Immunol. 1999;163(8):4224-4231).

In another example embodiment, a multimerizing peptide sequence is appended to the binding protein sequence. Sequences for a promoter and a terminator sequence are also appended to the sequence, and an IVTT reaction is used to produce self-assembled binding protein multimers. Binding protein multimers are subsequently used to stain bead-bound pMHC or cellular targets loaded with target or control peptide (e.g., pulsing TAP−/− cells with peptide, expressing peptides fused to an ER signal sequence, or transducing cells with single-chain pMHC), and binding protein multimer binding is analyzed with flow cytometry to determine on-target and off-target binding.

### Cell PMHC Library

In example embodiments, cell lines are generated that express a library of peptides presented on MHC molecules on the surface of cells (cellular peptide-display libraries). MHCI peptides can be presented on any cell line known in the art (e.g., pulsing TAP−/− cells with peptide, expressing peptides fused to an ER signal sequence, or transducing cells with single-chain pMHC). In preferred embodiments, TAP−/− cells are used. MHCII peptides can be presented on any antigen presenting cell line known in the art (e.g., dendritic cells, B-lymphocytes, macrophages, and other antigen-presenting cells). The cell library can be used to identify the peptides that a binding protein binds to, such that specificity for the target peptide and off-target binding can be determined. The binding protein can be expressed as a monomer or multimer. The binding protein can be labeled with a marker that can be used to sort cells. The marker can be a fluorescent marker. Sorted cells can be sequenced to determine the sequence of the bound peptides. The cell lines can be barcoded to indicate the peptide expressed by individual cells. Sequencing of the barcode in cells bound by the binding protein can be used to identify the peptides bound.

### Cytotoxicity and Suppression Assays

Cytotoxicity and suppression assays can be used to validate identified pMHC-targeting proteins. In example embodiments, the assay uses CAR T cells or bispecific antibodies incorporating a binding protein. In example embodiments, candidate binding proteins against pMHC presented on target cells can be evaluated by generating bispecific T cell engager (BITE) proteins including (1) a protein domain binding to CD3 or any T cell surface marker, (2) a flexible linker, and (3) the candidate binding protein, and incubating these BITES with CD8+ T cells and target cells encompassing (1) peptide-pulsed TAP−/− cells, (2) TAP−/− cells expressing peptides fused to an ER signal sequence, (3) cells transduced with single-chain pMHC, or (4) cells expressing proteins containing the antigen peptide and processed naturally via the native antigen presentation pathway, including tumor cell lines and patient-derived cells. CAR T cells with a binding protein incorporated into the CAR can be co-cultured with target cells expressing the target pMHC and CD8+ T cell cytotoxicity activity is determined by directly evaluating cytotoxicity or by analyzing cytokines released by T cells (e.g., IFN-γ, TNF-α). Suppression assays can be performed using CAR Treg cells with a binding protein incorporated into the CAR co-cultured with cytotoxic T cells specific for a target cell expressing the target pMHC and directly evaluating cytotoxicity or by analyzing cytokines released by T cells (e.g., IFN-γ, TNF-α) or the Treg cells (e.g., IL-10).

### Therapeutic Binding Proteins

Chimeric antigen receptors

In example embodiments, a binding protein is assembled into a chimeric antigen receptor (CAR) for targeting diseased cells with CAR T cells (e.g., cancer) or CAR Treg cells (e.g., autoimmune diseases or transplantation). In example embodiments, an antigen (such as a tumor antigen or self-antigen) to be targeted in adoptive cell therapy (ACT) (such as particularly CAR or TCR T-cell therapy) of a disease (such as particularly of tumor or cancer or autoimmune disease) may be targeted using a binding protein according to the present invention.

In example embodiments, T cell therapy may incorporate binding domains identified by the methods described herein. Various strategies may for example be employed to genetically modify T cells by altering the specificity of the T cell receptor (TCR) for example by introducing new TCR a and B chains with selected peptide specificity (see U.S. Pat. No. 8,697,854; PCT Patent Publications: WO2003020763, WO2004033685, WO2004044004, WO2005114215, WO2006000830, WO2008038002, WO2008039818, WO2004074322, WO2005113595, WO2006125962, WO2013166321, WO2013039889, WO2014018863, WO2014083173; U.S. Pat. No. 8,088,379).

As an alternative to, or addition to, TCR modifications, chimeric antigen receptors (CARs) may be used in order to generate immunoresponsive cells, such as T cells or natural killer cells (NK), specific for selected targets, such as malignant cells, with a wide variety of receptor chimera constructs having been described (see U.S. Pat. Nos. 5,843,728; 5,851,828; 5,912,170; 6,004,811; 6,284,240; 6,392,013; 6,410,014; 6,753,162; 8,211,422; and, PCT Publication WO9215322).

In general, CARs are comprised of an extracellular domain, a transmembrane domain, and an intracellular domain, wherein the extracellular domain comprises an antigen-binding domain that is specific for a predetermined target (see, e.g., Gong Y, Klein Wolterink RGJ, Wang J, Bos GMJ, Germeraad WTV. Chimeric antigen receptor natural killer (CAR-NK) cell design and engineering for cancer therapy. J Hematol Oncol. 2021;14(1):73; Guedan S, Calderon H, Posey A D Jr, Maus M V. Engineering and Design of Chimeric Antigen Receptors. Mol Ther Methods Clin Dev. 2018; 12:145-156; Petersen CT, Krenciute G. Next Generation CAR T Cells for the Immunotherapy of High-Grade Glioma. Front Oncol. 2019;9:69; and Lu H, Zhao X, Li Z, Hu Y, Wang H. From CAR-T Cells to CAR-NK Cells: A Developing Immunotherapy Method for Hematological Malignancies. Front Oncol. 2021). While the antigen-binding domain of a CAR is often an antibody or antibody fragment (e.g., a single chain variable fragment, scFv), the binding domain is not particularly limited so long as it results in specific recognition of a target. For example, in some embodiments, the antigen-binding domain may comprise a receptor, such that the CAR is capable of binding to the ligand of the receptor. Alternatively, the antigen-binding domain may comprise a ligand, such that the CAR is capable of binding the endogenous receptor of that ligand.

The antigen-binding domain of a CAR is generally separated from the transmembrane domain by a hinge or spacer. The spacer is also not particularly limited, and it is designed to provide the CAR with flexibility. For example, a spacer domain may comprise a portion of a human Fc domain, including a portion of the CH3 domain, or the hinge region of any immunoglobulin, such as IgA, IgD, IgE, IgG, or IgM, or variants thereof. Furthermore, the hinge region may be modified so as to prevent off-target binding by FcRs or other potential interfering objects. For example, the hinge may comprise an IgG4 Fc domain with or without a S228P, L235E, and/or N297Q mutation (according to Kabat numbering) in order to decrease binding to FcRs. Additional spacers/hinges include, but are not limited to, CD4, CD8, and CD28 hinge regions.

The transmembrane domain of a CAR may be derived either from a natural or from a synthetic source. Where the source is natural, the domain may be derived from any membrane bound or transmembrane protein. Transmembrane regions of particular use in this disclosure may be derived from CD8, CD28, CD3, CD45, CD4, CD5, CDS, CD9, CD 16, CD22, CD33, CD37, CD64, CD80, CD86, CD 134, CD137, CD 154, TCR. Alternatively, the transmembrane domain may be synthetic, in which case it will comprise predominantly hydrophobic residues such as leucine and valine. Preferably a triplet of phenylalanine, tryptophan and valine will be found at each end of a synthetic transmembrane domain. Optionally, a short oligo-or polypeptide linker, preferably between 2 and 10 amino acids in length may form the linkage between the transmembrane domain and the cytoplasmic signaling domain of the CAR. A glycine-serine doublet provides a particularly suitable linker.

Alternative CAR constructs may be characterized as belonging to successive generations. First-generation CARs typically consist of a single-chain variable fragment of an antibody specific for an antigen, for example comprising a VL linked to a VH of a specific antibody, linked by a flexible linker, for example by a CD8α hinge domain and a CD8α transmembrane domain, to the transmembrane and intracellular signaling domains of either CD3ζ or FcRγ (scFv-CD3ζ or scFv-FcRγ; see U.S. Pat. No. 7,741,465; U.S. Pat. No. 5,912,172; U.S. Pat. No. 5,906,936). Second-generation CARs incorporate the intracellular domains of one or more costimulatory molecules, such as CD28, OX40 (CD134), or 4-1BB (CD137) within the endodomain (for example scFv-CD28/OX40/4-1BB-CD3ζ; see U.S. Pat. Nos. 8,911,993; 8,916,381; 8,975,071; 9,101,584; 9,102,760; 9,102,761). Third-generation CARs include a combination of costimulatory endodomains, such a CD3ζ-chain, CD97, GDI la-CD18, CD2, ICOS, CD27, CD154, CDS, OX40, 4-1BB, CD2, CD7, LIGHT, LFA-1, NKG2C, B7-H3, CD30, CD40, PD-1, or CD28 signaling domains (for example scFv-CD28-4-1BB-CD3ζ or scFv-CD28-OX40-CD3ζ; see U.S. Pat. No. 8,906,682; U.S. Pat. No. 8,399,645; U.S. Pat. No. 5,686,281; PCT Publication No. WO2014134165; PCT Publication No. WO2012079000). In certain embodiments, the primary signaling domain comprises a functional signaling domain of a protein selected from the group consisting of CD3 zeta, CD3 gamma, CD3 delta, CD3 epsilon, common FcR gamma (FCERIG), FcR beta (Fc Epsilon R1b), CD79a, CD79b, Fc gamma RIIa, DAP10, and DAP12. In certain preferred embodiments, the primary signaling domain comprises a functional signaling domain of CD3ζ or FcRγ. In certain embodiments, the one or more costimulatory signaling domains comprise a functional signaling domain of a protein selected, each independently, from the group consisting of: CD27, CD28, 4-1BB (CD137), OX40, CD30, CD40, PD-1, ICOS, lymphocyte function-associated antigen-1 (LFA-1), CD2, CD7, LIGHT, NKG2C, B7-H3, a ligand that specifically binds with CD83, CDS, ICAM-1, GITR, BAFFR, HVEM (LIGHTR), SLAMF7, NKp80 (KLRF1), CD160, CD19, CD4, CD8 alpha, CD8 beta, IL2R beta, IL2R gamma, IL7R alpha, ITGA4, VLA1, CD49a, ITGA4, IA4, CD49D, ITGA6, VLA-6, CD49f, ITGAD, CD11d, ITGAE, CD103, ITGAL, CD11a, LFA-1, ITGAM, CD11b, ITGAX, CD11c, ITGB1, CD29, ITGB2, CD18, ITGB7, TNFR2, TRANCE/RANKL, DNAMI (CD226), SLAMF4 (CD244, 2B4), CD84, CD96 (Tactile), CEACAMI, CRTAM, Ly9 (CD229), CD160 (BY55), PSGL1, CD100 (SEMA4D), CD69, SLAMF6 (NTB-A, Ly108), SLAM (SLAMF1, CD150, IPO-3), BLAME (SLAMF8), SELPLG (CD162), LTBR, LAT, GADS, SLP-76, PAG/Cbp, NKp44, NKp30, NKp46, and NKG2D. In certain embodiments, the one or more costimulatory signaling domains comprise a functional signaling domain of a protein selected, each independently, from the group consisting of: 4-1BB, CD27, and CD28. In certain embodiments, a chimeric antigen receptor may have the design as described in U.S. Pat. No. 7,446,190, comprising an intracellular domain of CD3ζ chain (such as amino acid residues 52-163 of the human CD3 zeta chain, as shown in SEQ ID NO: 14 of U.S. Pat. No. 7,446,190), a signaling region from CD28 and an antigen-binding element (or portion or domain; such as scFv). The CD28 portion, when between the zeta chain portion and the antigen-binding element, may suitably include the transmembrane and signaling domains of CD28 (such as amino acid residues 114-220 of SEQ ID NO: 10, full sequence shown in SEQ ID NO: 6 of U.S. Pat. No. 7,446,190; these can include the following portion of CD28 as set forth in Genbank identifier NM_006139 (sequence version 1, 2 or 3): IEVMYPPPYLDNEKSNGTIIHVKGKHLCPSPLFPGPSKPFWVLVVVGGVLACYSLLVTVA FIIFWVRSKRSRLLHSDYMNMTPRRPGPTRKHYQPYAPPRDFAAYRS)) (SEQ ID NO: 1). Alternatively, when the zeta sequence lies between the CD28 sequence and the antigen-binding element, intracellular domain of CD28 can be used alone (such as amino sequence set forth in SEQ ID NO: 9 of U.S. Pat. No. 7,446,190). Hence, certain embodiments employ a CAR comprising (a) a zeta chain portion comprising the intracellular domain of human CD3ζ chain, (b) a costimulatory signaling region, and (c) an antigen-binding element (or portion or domain), wherein the costimulatory signaling region comprises the amino acid sequence encoded by SEQ ID NO: 6 of U.S. Pat. No. 7,446,190.

Alternatively, costimulation may be orchestrated by expressing CARs in antigen-specific T cells, chosen so as to be activated and expanded following engagement of their native αβTCR, for example by antigen on professional antigen-presenting cells, with attendant costimulation. In addition, additional engineered receptors may be provided on the immunoresponsive cells, for example to improve targeting of a T-cell attack and/or minimize side effects.

By means of an example and without limitation, Kochenderfer et al., (2009) J Immunother. 32 (7): 689-702 described anti-CD19 chimeric antigen receptors (CAR). FMC63-28Z CAR contained a single chain variable region moiety (scFv) recognizing CD19 derived from the FMC63 mouse hybridoma (described in Nicholson et al., (1997) Molecular Immunology 34:1157-1165), a portion of the human CD28 molecule, and the intracellular component of the human TCR-2 molecule. FMC63-CD828BBZ CAR contained the FMC63 scFv, the hinge and transmembrane regions of the CD8 molecule, the cytoplasmic portions of CD28 and 4-1BB, and the cytoplasmic component of the TCR-ζ molecule. The exact sequence of the CD28 molecule included in the FMC63-28Z CAR corresponded to Genbank identifier NM_006139; the sequence included all amino acids starting with the amino acid sequence IEVMYPPPY (SEQ ID NO: 2) and continuing all the way to the carboxy-terminus of the protein. To encode the anti-CD19 scFv component of the vector, the authors designed a DNA sequence which was based on a portion of a previously published CAR (Cooper et al., (2003) Blood 101: 1637-1644). This sequence encoded the following components in frame from the 5′ end to the 3′ end: an XhoI site, the human granulocyte-macrophage colony-stimulating factor (GM-CSF) receptor α-chain signal sequence, the FMC63 light chain variable region (as in Nicholson et al., supra), a linker peptide (as in Cooper et al., supra), the FMC63 heavy chain variable region (as in Nicholson et al., supra), and a NotI site. A plasmid encoding this sequence was digested with XhoI and NotI. To form the MSGV-FMC63-28Z retroviral vector, the XhoI and NotI-digested fragment encoding the FMC63 scFv was ligated into a second XhoI and NotI-digested fragment that encoded the MSGV retroviral backbone (as in Hughes et al., (2005) Human Gene Therapy 16: 457-472) as well as part of the extracellular portion of human CD28, the entire transmembrane and cytoplasmic portion of human CD28, and the cytoplasmic portion of the human TCR-ζ molecule (as in Maher et al., 2002) Nature Biotechnology 20: 70-75). The FMC63-28Z CAR is included in the KTE-C19 (axicabtagene ciloleucel) anti-CD19 CAR-T therapy product in development by Kite Pharma, Inc. for the treatment of inter alia patients with relapsed/refractory aggressive B-cell non-Hodgkin lymphoma (NHL). Accordingly, in certain embodiments, cells intended for adoptive cell therapies, more particularly immunoresponsive cells such as T cells, may express the FMC63-28Z CAR as described by Kochenderfer et al. (supra). Hence, in certain embodiments, cells intended for adoptive cell therapies, more particularly immunoresponsive cells such as T cells, may comprise a CAR comprising an extracellular antigen-binding element (or portion or domain; such as scFv) that specifically binds to an antigen, an intracellular signaling domain comprising an intracellular domain of a CD3ζ chain, and a costimulatory signaling region comprising a signaling domain of CD28. Preferably, the CD28 amino acid sequence is as set forth in Genbank identifier NM_006139 (sequence version 1, 2 or 3) starting with the amino acid sequence IEVMYPPPY (SEQ ID NO: 2) and continuing all the way to the carboxy-terminus of the protein. The sequence is reproduced herein:

IEVMYPPPYLDNEKSNGTIIHVKGKHLCPSPLFPGPSKPFWVLVVVGGVLACYSLLVTVA FIIFWVRSKRSRLLHSDYMNMTPRRPGPTRKHYQPYAPPRDFAAYRS (SEQ ID NO: 1). Preferably, the antigen is CD19, more preferably the antigen-binding element is an anti-CD19 scFv, even more preferably the anti-CD19 scFv as described by Kochenderfer et al. (supra).

Additional anti-CD19 CARs are further described in WO2015187528. More particularly Example 1 and Table 1 of WO2015187528, incorporated by reference herein, demonstrate the generation of anti-CD19 CARs based on a fully human anti-CD19 monoclonal antibody (47G4, as described in U.S. Pat. No. 20,100,104509) and murine anti-CD19 monoclonal antibody (as described in Nicholson et al. and explained above). Various combinations of a signal sequence (human CD8-alpha or GM-CSF receptor), extracellular and transmembrane regions (human CD8-alpha) and intracellular T-cell signaling domains (CD28-CD3ζ; 4-1BB-CD3ζ; CD27-CD3ζ; CD28-CD27-CD3ζ, 4-1BB-CD27-CD3ζ; CD27-4-1BB-CD3ζ; CD28-CD27-FcεRI gamma chain; or CD28-FcεRI gamma chain) were disclosed. Hence, in certain embodiments, cells intended for adoptive cell therapies, more particularly immunoresponsive cells such as T cells, may comprise a CAR comprising an extracellular antigen-binding element that specifically binds to an antigen, an extracellular and transmembrane region as set forth in Table 1 of WO2015187528 and an intracellular T-cell signaling domain as set forth in Table 1 of WO2015187528. Preferably, the antigen is CD19, more preferably the antigen-binding element is an anti-CD19 scFv, even more preferably the mouse or human anti-CD19 scFv as described in Example 1 of WO2015187528. In certain embodiments, the CAR comprises, consists essentially of or consists of an amino acid sequence of SEQ ID NO: 1, SEQ ID NO: 2, SEQ ID NO: 3, SEQ ID NO: 4, SEQ ID NO: 5, SEQ ID NO: 6, SEQ ID NO: 7, SEQ ID NO: 8, SEQ ID NO: 9, SEQ ID NO: 10, SEQ ID NO: 11, SEQ ID NO: 12, or SEQ ID NO: 13 as set forth in Table 1 of WO2015187528.

By means of an example and without limitation, chimeric antigen receptor that recognizes the CD70 antigen is described in WO2012058460A2 (see also, Park et al., CD70 as a target for chimeric antigen receptor T cells in head and neck squamous cell carcinoma, Oral Oncol. 2018 March;78:145-150; and Jin et al., CD70, a novel target of CAR T-cell therapy for gliomas, Neuro Oncol. 2018 Jan. 10;20(1):55-65). CD70 is expressed by diffuse large B-cell and follicular lymphoma and also by the malignant cells of Hodgkins lymphoma, Waldenstrom's macroglobulinemia and multiple myeloma, and by HTLV-1-and EBV-associated malignancies. (Agathanggelou et al. Am.J.Pathol. 1995;147:1152-1160; Hunter et al., Blood 2004;104:4881. 26; Lens et al., J Immunol. 2005;174:6212-6219; Baba et al., J Virol. 2008;82:3843-3852.) In addition, CD70 is expressed by non-hematological malignancies such as renal cell carcinoma and glioblastoma. (Junker et al., J Urol. 2005;173:2150-2153; Chahlavi et al., Cancer Res 2005;65:5428-5438) Physiologically, CD70 expression is transient and restricted to a subset of highly activated T, B, and dendritic cells.

By means of an example and without limitation, chimeric antigen receptor that recognizes BCMA has been described (see, e.g., US20160046724A1; WO2016014789A2; WO2017211900A1; WO2015158671A1; US20180085444A1; WO2018028647A1; US20170283504A1; and WO2013154760A1).

In certain embodiments, the immune cell may, in addition to a CAR or exogenous TCR as described herein, further comprise a chimeric inhibitory receptor (inhibitory CAR) that specifically binds to a second target antigen and is capable of inducing an inhibitory or immunosuppressive or repressive signal to the cell upon recognition of the second target antigen. In certain embodiments, the chimeric inhibitory receptor comprises an extracellular antigen-binding element (or portion or domain) configured to specifically bind to a target antigen, a transmembrane domain, and an intracellular immunosuppressive or repressive signaling domain. In certain embodiments, the second target antigen is an antigen that is not expressed on the surface of a cancer cell or infected cell or the expression of which is downregulated on a cancer cell or an infected cell. In certain embodiments, the second target antigen is an MHC-class I molecule. In certain embodiments, the intracellular signaling domain comprises a functional signaling portion of an immune checkpoint molecule, such as for example PD-1 or CTLA4. Advantageously, the inclusion of such inhibitory CAR reduces the chance of the engineered immune cells attacking non-target (e.g., non-cancer) tissues.

Alternatively, T-cells expressing CARs may be further modified to reduce or eliminate expression of endogenous TCRs in order to reduce off-target effects. Reduction or elimination of endogenous TCRs can reduce off-target effects and increase the effectiveness of the T cells (U.S. Pat. No. 9,181,527). T cells stably lacking expression of a functional TCR may be produced using a variety of approaches. T cells internalize, sort, and degrade the entire T cell receptor as a complex, with a half-life of about 10 hours in resting T cells and 3 hours in stimulated T cells (von Essen, M. et al. 2004. J. Immunol. 173:384-393). Proper functioning of the TCR complex requires the proper stoichiometric ratio of the proteins that compose the TCR complex. TCR function also requires two functioning TCR zeta proteins with ITAM motifs. The activation of the TCR upon engagement of its MHC-peptide ligand requires the engagement of several TCRs on the same T cell, which all must signal properly. Thus, if a TCR complex is destabilized with proteins that do not associate properly or cannot signal optimally, the T cell will not become activated sufficiently to begin a cellular response.

Accordingly, in some embodiments, TCR expression may eliminated using RNA interference (e.g., shRNA, siRNA, miRNA, etc.), CRISPR, or other methods that target the nucleic acids encoding specific TCRs (e.g., TCR-α and TCR-β) and/or CD3 chains in primary T cells. By blocking expression of one or more of these proteins, the T cell will no longer produce one or more of the key components of the TCR complex, thereby destabilizing the TCR complex and preventing cell surface expression of a functional TCR.

In some instances, CAR may also comprise a switch mechanism for controlling expression and/or activation of the CAR. For example, a CAR may comprise an extracellular, transmembrane, and intracellular domain, in which the extracellular domain comprises a target-specific binding element that comprises a label, binding domain, or tag that is specific for a molecule other than the target antigen that is expressed on or by a target cell. In such embodiments, the specificity of the CAR is provided by a second construct that comprises a target antigen binding domain (e.g., an scFv or a bispecific antibody that is specific for both the target antigen and the label or tag on the CAR) and a domain that is recognized by or binds to the label, binding domain, or tag on the CAR. See, e.g., WO 2013/044225, WO 2016/000304, WO 2015/057834, WO 2015/057852, WO 2016/070061, U.S. Pat. No. 9,233,125, US 2016/0129109. In this way, a T-cell that expresses the CAR can be administered to a subject, but the CAR cannot bind its target antigen until the second composition comprising an antigen-specific binding domain is administered.

Alternative switch mechanisms include CARs that require multimerization in order to activate their signaling function (see, e.g., US 2015/0368342, US 2016/0175359, US 2015/0368360) and/or an exogenous signal, such as a small molecule drug (US 2016/0166613, Yung et al., Science, 2015), in order to elicit a T-cell response. Some CARs may also comprise a “suicide switch” to induce cell death of the CAR T-cells following treatment (Buddee et al., PLoS One, 2013) or to downregulate expression of the CAR following binding to the target antigen (WO 2016/011210).

Alternative techniques may be used to transform target immunoresponsive cells, such as protoplast fusion, lipofection, transfection or electroporation. A wide variety of vectors may be used, such as retroviral vectors, lentiviral vectors, adenoviral vectors, adeno-associated viral vectors, plasmids, or transposons, such as a Sleeping Beauty transposon (see U.S. Pat. Nos. 6,489,458; 7,148,203; 7,160,682; 7,985,739; 8,227,432), may be used to introduce CARs, for example using 2nd generation antigen-specific CARs signaling through CD3ζ and either CD28 or CD137. Viral vectors may for example include vectors based on HIV, SV40, EBV, HSV or BPV. In certain embodiments, inducible gene switches are used to regulate expression of a CAR or TCR (see, e.g., Chakravarti, Deboki et al. “Inducible Gene Switches with Memory in Human T Cells for Cellular Immunotherapy.” ACS synthetic biology vol. 8,8 (2019): 1744-1754).

Cells that are targeted for transformation may for example include T cells, Natural Killer (NK) cells, cytotoxic T lymphocytes (CTL), regulatory T cells, human embryonic stem cells, tumor-infiltrating lymphocytes (TIL) or a pluripotent stem cell from which lymphoid cells may be differentiated. T cells expressing a desired CAR may for example be selected through co-culture with γ-irradiated activating and propagating cells (AaPC), which co-express the cancer antigen and co-stimulatory molecules. The engineered CAR T-cells may be expanded, for example by co-culture on AaPC in presence of soluble factors, such as IL-2 and IL-21. This expansion may for example be carried out so as to provide memory CAR+ T cells (which may for example be assayed by non-enzymatic digital array and/or multi-panel flow cytometry). In this way, CAR T cells may be provided that have specific cytotoxic activity against antigen-bearing tumors (optionally in conjunction with production of desired chemokines such as interferon-γ). CAR T cells of this kind may for example be used in animal models, for example to treat tumor xenografts.

In certain embodiments, ACT includes co-transferring CD4+ Th1 cells and CD8+ CTLs to induce a synergistic antitumour response (see, e.g., Li et al., Adoptive cell therapy with CD4+ T helper 1 cells and CD8+ cytotoxic T cells enhances complete rejection of an established tumour, leading to generation of endogenous memory responses to non-targeted tumour epitopes. Clin Transl Immunology. 2017 October; 6(10): e160).

In certain embodiments, antigen specificity can be conferred to Tregs by engineering the expression of transgenic T-cell receptor (TCR) or chimeric antigen receptor (CAR), such as to modulate immune responses in organ transplant and autoimmune diseases (see, e.g., Arjomandnejad M, Kopec AL, Keeler AM. CAR-T Regulatory (CAR-Treg) Cells: Engineering and Applications. Biomedicines. 2022;10(2):287). Regulatory T cells (Tregs) are a T-cell subset known for their immunomodulatory function. Expression of CD4, CD25, and the master transcription factor, forkhead box P3 (FOXP3), are the main characteristic markers of conventional Tregs. However, other regulatory immune cells with different properties such as CD8+ Tregs, or type 1 regulatory T cells (Tr1) have been described. Id. Tregs are divided into “natural” Tregs that develop in the thymus or “induced” Tregs that are generated in the periphery. Id. Regulatory T cells suppress immune responses through multiple mechanisms including direct interaction with other immune cells or by producing immunosuppressive cytokines such as interleukin-10 (IL-10) and Transforming growth factor beta (TGF-β. Id. Directing Tregs towards a desired antigen may boost the overall response and lower the risk of broad and systemic immunosuppression or generation of an inflammatory response. Id.

In certain embodiments, Th17 cells are transferred to a subject in need thereof. Th17 cells have been reported to directly eradicate melanoma tumors in mice to a greater extent than Th1 cells (Muranski P, et al., Tumor-specific Th17-polarized cells eradicate large established melanoma. Blood. 2008 Jul. 15; 112(2):362-73; and Martin-Orozco N, et al., T helper 17 cells promote cytotoxic T cell activation in tumor immunity. Immunity. 2009 Nov. 20; 31(5):787-98). Those studies involved an adoptive T cell transfer (ACT) therapy approach, which takes advantage of CD4+ T cells that express a TCR recognizing tyrosinase tumor antigen. Exploitation of the TCR leads to rapid expansion of Th17 populations to large numbers ex vivo for reinfusion into the autologous tumor-bearing hosts.

Unlike T-cell receptors (TCRs) that are MHC restricted, CARs can potentially bind any cell surface-expressed antigen and can thus be more universally used to treat patients (see Irving et al., Engineering Chimeric Antigen Receptor T-Cells for Racing in Solid Tumors: Don't Forget the Fuel, Front. Immunol., 3 Apr. 2017, doi.org/10.3389/fimmu.2017.00267). In certain embodiments, in the absence of endogenous T-cell infiltrate (e.g., due to aberrant antigen processing and presentation), which precludes the use of TIL therapy and immune checkpoint blockade, the transfer of CAR T-cells may be used to treat patients (see, e.g., Hinrichs CS, Rosenberg SA. Exploiting the curative potential of adoptive T-cell therapy for cancer. Immunol Rev (2014) 257(1):56-71. doi: 10.1111/imr.12132).

Approaches such as the foregoing may be adapted to provide methods of treating and/or increasing survival of a subject having a disease, such as a neoplasia, for example by administering an effective amount of an immunoresponsive cell comprising an antigen recognizing receptor that binds a selected antigen, wherein the binding activates the immunoresponsive cell, thereby treating or preventing the disease (such as a neoplasia, a pathogen infection, an autoimmune disorder, or an allogeneic transplant reaction).

To guard against possible adverse reactions, engineered immunoresponsive cells may be equipped with a transgenic safety switch, in the form of a transgene that renders the cells vulnerable to exposure to a specific signal. For example, the herpes simplex viral thymidine kinase (TK) gene may be used in this way, for example by introduction into allogeneic T lymphocytes used as donor lymphocyte infusions following stem cell transplantation (Greco, et al., Improving the safety of cell therapy with the TK-suicide gene. Front. Pharmacol. 2015; 6:95). In such cells, administration of a nucleoside prodrug such as ganciclovir or acyclovir causes cell death. Alternative safety switch constructs include inducible caspase 9, for example triggered by administration of a small-molecule dimerizer that brings together two nonfunctional icasp9 molecules to form the active enzyme. A wide variety of alternative approaches to implementing cellular proliferation controls have been described (see U.S. Patent Publication No. 20130071414; PCT Patent Publication WO2011146862; PCT Patent Publication WO2014011987; PCT Patent Publication WO2013040371; Zhou et al. BLOOD, 2014, 123/25:3895-3905; Di Stasi et al., The New England Journal of Medicine 2011; 365:1673-1683; Sadelain M, The New England Journal of Medicine 2011; 365:1735-173; Ramos et al., Stem Cells 28(6):1107-15 (2010)).

In a further refinement of adoptive therapies, genome editing may be used to tailor immunoresponsive cells to alternative implementations, for example providing edited CAR T cells (see Poirot et al., 2015, Multiplex genome edited T-cell manufacturing platform for “off-the-shelf” adoptive T-cell immunotherapies, Cancer Res 75 (18): 3853; Ren et al., 2017, Multiplex genome editing to generate universal CAR T cells resistant to PD1 inhibition, Clin Cancer Res. 2017 May 1;23(9):2255-2266. doi: 10.1158/1078-0432.CCR-16-1300. Epub 2016 Nov. 4; Qasim et al., 2017, Molecular remission of infant B-ALL after infusion of universal TALEN gene-edited CAR T cells, Sci Transl Med. 2017 Jan. 25;9(374); Legut, et al., 2018, CRISPR-mediated TCR replacement generates superior anticancer transgenic T cells. Blood, 131(3), 311-322; Georgiadis et al., Long Terminal Repeat CRISPR-CAR-Coupled “Universal” T Cells Mediate Potent Anti-leukemic Effects, Molecular Therapy, In Press, Corrected Proof, Available online 6 Mar. 2018; and Roth, T.L. Editing of Endogenous Genes in Cellular Immunotherapies. Curr Hematol Malig Rep 15, 235-240 (2020)). Cells may be edited using any CRISPR system and method of use thereof as described herein. CRISPR systems may be delivered to an immune cell by any method described herein. In preferred embodiments, cells are edited ex vivo and transferred to a subject in need thereof. Immunoresponsive cells, CAR T cells or any cells used for adoptive cell transfer may be edited. Editing may be performed for example to insert or knock-in an exogenous gene, such as an exogenous gene encoding a CAR or a TCR, at a preselected locus in a cell (e.g. TRAC locus); to eliminate potential alloreactive T-cell receptors (TCR) or to prevent inappropriate pairing between endogenous and exogenous TCR chains, such as to knock-out or knock-down expression of an endogenous TCR in a cell; to disrupt the target of a chemotherapeutic agent in a cell; to block an immune checkpoint, such as to knock-out or knock-down expression of an immune checkpoint protein or receptor in a cell; to knock-out or knock-down expression of other gene or genes in a cell, the reduced expression or lack of expression of which can enhance the efficacy of adoptive therapies using the cell; to knock-out or knock-down expression of an endogenous gene in a cell, said endogenous gene encoding an antigen targeted by an exogenous CAR or TCR; to knock-out or knock-down expression of one or more MHC constituent proteins in a cell; to activate a T cell; to modulate cells such that the cells are resistant to exhaustion or dysfunction; and/or increase the differentiation and/or proliferation of functionally exhausted or dysfunctional CD8+ T-cells (see PCT Patent Publications: WO2013176915, WO2014059173, WO2014172606, WO2014184744, and WO2014191128).

In certain embodiments, editing may result in inactivation of a gene. By inactivating a gene, it is intended that the gene of interest is not expressed in a functional protein form. In a particular embodiment, the CRISPR system specifically catalyzes cleavage in one targeted gene thereby inactivating said targeted gene. The nucleic acid strand breaks caused are commonly repaired through the distinct mechanisms of homologous recombination or non-homologous end joining (NHEJ). However, NHEJ is an imperfect repair process that often results in changes to the DNA sequence at the site of the cleavage. Repair via non-homologous end joining (NHEJ) often results in small insertions or deletions (Indel) and can be used for the creation of specific gene knockouts. Cells in which a cleavage induced mutagenesis event has occurred can be identified and/or selected by well-known methods in the art. In certain embodiments, homology directed repair (HDR) is used to concurrently inactivate a gene (e.g., TRAC) and insert an endogenous TCR or CAR into the inactivated locus.

Hence, in certain embodiments, editing of cells (such as by CRISPR/Cas), particularly cells intended for adoptive cell therapies, more particularly immunoresponsive cells such as T cells, may be performed to insert or knock-in an exogenous gene, such as an exogenous gene encoding a CAR or a TCR, at a preselected locus in a cell. Conventionally, nucleic acid molecules encoding CARs or TCRs are transfected or transduced to cells using randomly integrating vectors, which, depending on the site of integration, may lead to clonal expansion, oncogenic transformation, variegated transgene expression and/or transcriptional silencing of the transgene. Directing of transgene(s) to a specific locus in a cell can minimize or avoid such risks and advantageously provide for uniform expression of the transgene(s) by the cells. Without limitation, suitable ‘safe harbor’ loci for directed transgene integration include CCR5 or AAVS1. Homology-directed repair (HDR) strategies are known and described elsewhere in this specification allowing to insert transgenes into desired loci (e.g., TRAC locus).

Further suitable loci for insertion of transgenes, in particular CAR or exogenous TCR transgenes, include without limitation loci comprising genes coding for constituents of endogenous T-cell receptor, such as T-cell receptor alpha locus (TRA) or T-cell receptor beta locus (TRB), for example T-cell receptor alpha constant (TRAC) locus, T-cell receptor beta constant 1 (TRBC1) locus or T-cell receptor beta constant 2 (TRBC1) locus. Advantageously, insertion of a transgene into such locus can simultaneously achieve expression of the transgene, potentially controlled by the endogenous promoter, and knock-out expression of the endogenous TCR. This approach has been exemplified in Eyquem et al., (2017) Nature 543: 113-117, wherein the authors used CRISPR/Cas9 gene editing to knock-in a DNA molecule encoding a CD19-specific CAR into the TRAC locus downstream of the endogenous promoter; the CAR-T cells obtained by CRISPR were significantly superior in terms of reduced tonic CAR signaling and exhaustion.

T cell receptors (TCR) are cell surface receptors that participate in the activation of T cells in response to the presentation of antigen. The TCR is generally made from two chains, α and β, which assemble to form a heterodimer and associates with the CD3-transducing subunits to form the T cell receptor complex present on the cell surface. Each α and β chain of the TCR consists of an immunoglobulin-like N-terminal variable (V) and constant (C) region, a hydrophobic transmembrane domain, and a short cytoplasmic region. As for immunoglobulin molecules, the variable region of the α and β chains are generated by V(D)J recombination, creating a large diversity of antigen specificities within the population of T cells. However, in contrast to immunoglobulins that recognize intact antigen, T cells are activated by processed peptide fragments in association with an MHC molecule, introducing an extra dimension to antigen recognition by T cells, known as MHC restriction. Recognition of MHC disparities between the donor and recipient through the T cell receptor leads to T cell proliferation and the potential development of graft versus host disease (GVHD). The inactivation of TCRα or TCRβ can result in the elimination of the TCR from the surface of T cells preventing recognition of alloantigen and thus GVHD. However, TCR disruption generally results in the elimination of the CD3 signaling component and alters the means of further T cell expansion.

Hence, in certain embodiments, editing of cells (such as by CRISPR/Cas), particularly cells intended for adoptive cell therapies, more particularly immunoresponsive cells such as T cells, may be performed to knock-out or knock-down expression of an endogenous TCR in a cell. For example, NHEJ-based or HDR-based gene editing approaches can be employed to disrupt the endogenous TCR alpha and/or beta chain genes. For example, gene editing system or systems, such as CRISPR/Cas system or systems, can be designed to target a sequence found within the TCR beta chain conserved between the beta 1 and beta 2 constant region genes (TRBC1 and TRBC2) and/or to target the constant region of the TCR alpha chain (TRAC) gene.

Allogeneic cells are rapidly rejected by the host immune system. It has been demonstrated that, allogeneic leukocytes present in non-irradiated blood products will persist for no more than 5 to 6 days (Boni, Muranski et al. 2008 Blood 1;112(12):4746-54). Thus, to prevent rejection of allogeneic cells, the host's immune system usually has to be suppressed to some extent. However, in the case of adoptive cell transfer the use of immunosuppressive drugs also have a detrimental effect on the introduced therapeutic T cells. Therefore, to effectively use an adoptive immunotherapy approach in these conditions, the introduced cells would need to be resistant to the immunosuppressive treatment. Thus, in a particular embodiment, the present invention further comprises a step of modifying T cells to make them resistant to an immunosuppressive agent, preferably by inactivating at least one gene encoding a target for an immunosuppressive agent. An immunosuppressive agent is an agent that suppresses immune function by one of several mechanisms of action. An immunosuppressive agent can be, but is not limited to a calcineurin inhibitor, a target of rapamycin, an interleukin-2 receptor α-chain blocker, an inhibitor of inosine monophosphate dehydrogenase, an inhibitor of dihydrofolic acid reductase, a corticosteroid, or an immunosuppressive antimetabolite. The present invention allows conferring immunosuppressive resistance to T cells for immunotherapy by inactivating the target of the immunosuppressive agent in T cells. As non-limiting examples, targets for an immunosuppressive agent can be a receptor for an immunosuppressive agent such as: CD52, glucocorticoid receptor (GR), a FKBP family gene member and a cyclophilin family gene member.

In certain embodiments, editing of cells (such as by CRISPR/Cas), particularly cells intended for adoptive cell therapies, more particularly immunoresponsive cells such as T cells, may be performed to block an immune checkpoint, such as to knock-out or knock-down expression of an immune checkpoint protein or receptor in a cell. Immune checkpoints are inhibitory pathways that slow down or stop immune reactions and prevent excessive tissue damage from uncontrolled activity of immune cells. In certain embodiments, the immune checkpoint targeted is the programmed death-1 (PD-1 or CD279) gene (PDCD1) (see, e.g., Rupp L J, Schumann K, Roybal K T, et al. CRISPR/Cas9-mediated PD-1 disruption enhances anti-tumor efficacy of human chimeric antigen receptor T cells. Sci Rep. 2017;7(1):737). In other embodiments, the immune checkpoint targeted is cytotoxic T-lymphocyte-associated antigen (CTLA-4). In additional embodiments, the immune checkpoint targeted is another member of the CD28 and CTLA4 Ig superfamily such as BTLA, LAG3, ICOS, PDL1 or KIR. In further additional embodiments, the immune checkpoint targeted is a member of the TNFR superfamily such as CD40, OX40, CD137, GITR, CD27 or TIM-3.

Additional immune checkpoints include Src homology 2 domain-containing protein tyrosine phosphatase 1 (SHP-1) (Watson HA, et al., SHP-1: the next checkpoint target for cancer immunotherapy? Biochem Soc Trans. 2016 Apr. 15;44(2):356-62). SHP-1 is a widely expressed inhibitory protein tyrosine phosphatase (PTP). In T-cells, it is a negative regulator of antigen-dependent activation and proliferation. It is a cytosolic protein, and therefore not amenable to antibody-mediated therapies, but its role in activation and proliferation makes it an attractive target for genetic manipulation in adoptive transfer strategies, such as chimeric antigen receptor (CAR) T cells. Immune checkpoints may also include T cell immunoreceptor with Ig and ITIM domains (TIGIT/Vstm3/WUCAM/VSIG9) and VISTA (Le Mercier I, et al., (2015) Beyond CTLA-4 and PD-1, the generation Z of negative checkpoint regulators. Front. Immunol. 6:418).

WO2014172606 relates to the use of MT1 and/or MT2 inhibitors to increase proliferation and/or activity of exhausted CD8+ T-cells and to decrease CD8+ T-cell exhaustion (e.g., decrease functionally exhausted or unresponsive CD8+ immune cells). In certain embodiments, metallothioneins are targeted by gene editing in adoptively transferred T cells.

In certain embodiments, targets of gene editing may be at least one targeted locus involved in the expression of an immune checkpoint protein. Such targets may include, but are not limited to CTLA4, PPP2CA, PPP2CB, PTPN6, PTPN22, PDCD1, ICOS (CD278), PDL1, KIR, LAG3, HAVCR2, BTLA, CD160, TIGIT, CD96, CRTAM, LAIR1, SIGLEC7, SIGLEC9, CD244 (2B4), TNFRSF10B, TNFRSF10A, CASP8, CASP10, CASP3, CASP6, CASP7, FADD, FAS, TGFBRII, TGFRBRI, SMAD2, SMAD3, SMAD4, SMAD10, SKI, SKIL, TGIF1, IL1ORA, IL10RB, HMOX2, IL6R, IL6ST, EIF2AK4, CSK, PAG1, SIT1, FOXP3, PRDM1, BATF, VISTA, GUCY1A2, GUCY1A3, GUCY1B2, GUCY1B3, MT1, MT2, CD40, OX40, CD137, GITR, CD27, SHP-1, TIM-3, CEACAM-1, CEACAM-3, or CEACAM-5. In preferred embodiments, the gene locus involved in the expression of PD-1 or CTLA-4 genes is targeted. In other preferred embodiments, combinations of genes are targeted, such as but not limited to PD-1 and TIGIT.

By means of an example and without limitation, WO2016196388 concerns an engineered T cell comprising (a) a genetically engineered antigen receptor that specifically binds to an antigen, which receptor may be a CAR; and (b) a disrupted gene encoding a PD-L1, an agent for disruption of a gene encoding a PD-L1, and/or disruption of a gene encoding PD-L1, wherein the disruption of the gene may be mediated by a gene editing nuclease, a zinc finger nuclease (ZFN), CRISPR/Cas9 and/or TALEN. WO2015142675 relates to immune effector cells comprising a CAR in combination with an agent (such as CRISPR, TALEN or ZFN) that increases the efficacy of the immune effector cells in the treatment of cancer, wherein the agent may inhibit an immune inhibitory molecule, such as PD1, PD-L1, CTLA-4, TIM-3, LAG-3, VISTA, BTLA, TIGIT, LAIR1, CD160, 2B4, TGFR beta, CEACAM-1, CEACAM-3, or CEACAM-5. Ren et al., (2017) Clin Cancer Res 23 (9) 2255-2266 performed lentiviral delivery of CAR and electro-transfer of Cas9 mRNA and gRNAs targeting endogenous TCR, β-2 microglobulin (B2M) and PD1 simultaneously, to generate gene-disrupted allogeneic CAR T cells deficient of TCR, HLA class I molecule and PD1.

In certain embodiments, cells may be engineered to express a CAR, wherein expression and/or function of methylcytosine dioxygenase genes (TET1, TET2 and/or TET3) in the cells has been reduced or eliminated, such as by CRISPR, ZNF or TALEN (for example, as described in WO201704916).

In certain embodiments, editing of cells (such as by CRISPR/Cas), particularly cells intended for adoptive cell therapies, more particularly immunoresponsive cells such as T cells, may be performed to knock-out or knock-down expression of an endogenous gene in a cell, said endogenous gene encoding an antigen targeted by an exogenous CAR or TCR, thereby reducing the likelihood of targeting of the engineered cells. In certain embodiments, the targeted antigen may be one or more antigen selected from the group consisting of CD38, CD138, CS-1, CD33, CD26, CD30, CD53, CD92, CD100, CD148, CD150, CD200, CD261, CD262, CD362, human telomerase reverse transcriptase (hTERT), survivin, mouse double minute 2 homolog (MDM2), cytochrome P450 1B1 (CYP1B), HER2/neu, Wilms' tumor gene 1 (WT1), livin, alphafetoprotein (AFP), carcinoembryonic antigen (CEA), mucin 16 (MUC16), MUC1, prostate-specific membrane antigen (PSMA), p53, cyclin (D1), B cell maturation antigen (BCMA), transmembrane activator and CAML Interactor (TACI), and B-cell activating factor receptor (BAFF-R) (for example, as described in WO2016011210 and WO2017011804).

In certain embodiments, editing of cells (such as by CRISPR/Cas), particularly cells intended for adoptive cell therapies, more particularly immunoresponsive cells such as T cells, may be performed to knock-out or knock-down expression of one or more MHC constituent proteins, such as one or more HLA proteins and/or beta-2 microglobulin (B2M), in a cell, whereby rejection of non-autologous (e.g., allogeneic) cells by the recipient's immune system can be reduced or avoided. In preferred embodiments, one or more HLA class I proteins, such as HLA-A, B and/or C, and/or B2M may be knocked-out or knocked-down. Preferably, B2M may be knocked-out or knocked-down. By means of an example, Ren et al., (2017) Clin Cancer Res 23 (9) 2255-2266 performed lentiviral delivery of CAR and electro-transfer of Cas9 mRNA and gRNAs targeting endogenous TCR, β-2 microglobulin (B2M) and PD1 simultaneously, to generate gene-disrupted allogeneic CAR T cells deficient of TCR, HLA class I molecule and PD1.

In other embodiments, at least two genes are edited. Pairs of genes may include, but are not limited to PD1 and TCRα, PD1 and TCRβ, CTLA-4 and TCRα, CTLA-4 and TCRβ, LAG3 and TCRα, LAG3 and TCRβ, Tim3 and TCRα, Tim3 and TCRβ, BTLA and TCRα, BTLA and TCRβ, BY55 and TCRα, BY55 and TCRβ, TIGIT and TCRα, TIGIT and TCRβ, B7H5 and TCRα, B7H5 and TCRβ, LAIR1 and TCRα, LAIR1 and TCRβ, SIGLEC10 and TCRα, SIGLEC10 and TCRβ, 2B4 and TCRα, 2B4 and TCRβ, B2M and TCRα, B2M and TCRβ.

In certain embodiments, a cell may be multiply edited (multiplex genome editing) as taught herein to (1) knock-out or knock-down expression of an endogenous TCR (for example, TRBC1, TRBC2 and/or TRAC), (2) knock-out or knock-down expression of an immune checkpoint protein or receptor (for example PD1, PD-L1 and/or CTLA4); and (3) knock-out or knock-down expression of one or more MHC constituent proteins (for example, HLA-A, B and/or C, and/or B2M, preferably B2M).

In certain embodiments, T cells comprising a CAR or an exogenous TCR, may be manufactured as described in WO2015120096, by a method comprising: enriching a population of lymphocytes obtained from a donor subject; stimulating the population of lymphocytes with one or more T-cell stimulating agents to produce a population of activated T cells, wherein the stimulation is performed in a closed system using serum-free culture medium; transducing the population of activated T cells with a viral vector comprising a nucleic acid molecule which encodes the CAR or TCR, using a single cycle transduction to produce a population of transduced T cells, wherein the transduction is performed in a closed system using serum-free culture medium; and expanding the population of transduced T cells for a predetermined time to produce a population of engineered T cells, wherein the expansion is performed in a closed system using serum-free culture medium. In certain embodiments, T cells comprising a CAR or an exogenous TCR, may be manufactured as described in WO2015120096, by a method comprising: obtaining a population of lymphocytes; stimulating the population of lymphocytes with one or more stimulating agents to produce a population of activated T cells, wherein the stimulation is performed in a closed system using serum-free culture medium; transducing the population of activated T cells with a viral vector comprising a nucleic acid molecule which encodes the CAR or TCR, using at least one cycle transduction to produce a population of transduced T cells, wherein the transduction is performed in a closed system using serum-free culture medium; and expanding the population of transduced T cells to produce a population of engineered T cells, wherein the expansion is performed in a closed system using serum-free culture medium. The predetermined time for expanding the population of transduced T cells may be 3 days. The time from enriching the population of lymphocytes to producing the engineered T cells may be 6 days. The closed system may be a closed bag system. Further provided is population of T cells comprising a CAR or an exogenous TCR obtainable or obtained by said method, and a pharmaceutical composition comprising such cells.

### Bi-Specific Antigen-Binding Constructs

In example embodiments, a binding protein is assembled into bi-specific antigen-binding constructs, e.g., bi-specific antibodies (bsAb) or a bispecific engager molecule, such as bispecific T cell engagers (BiTEs), that bind two antigens (see, e.g., Suurs et al., A review of bispecific antibodies and antibody constructs in oncology and clinical challenges. Pharmacol Ther. 2019 September;201:103-119; and Huehls, et al., Bispecific T cell engagers for cancer immunotherapy. Immunol Cell Biol. 2015 March; 93(3): 290-296). The bi-specific antigen-binding construct includes two antigen-binding polypeptide constructs, e.g., antigen binding domains, wherein at least one polypeptide construct specifically binds to a tumor surface presented peptide. In some embodiments, the antigen-binding construct is derived using known antibodies or antigen-binding constructs. In some embodiments, the antigen-binding polypeptide constructs comprise two antigen binding domains that comprise antibody fragments. In some embodiments, the first antigen binding domain and second antigen binding domain each independently comprises an antibody fragment selected from the group of: an scFv, a Fab, and an Fc domain. The antigen binding domains may be the same format or different formats from each other (e.g., scFv and VHH). For example, in some embodiments, the antigen-binding polypeptide constructs comprise a first antigen binding domain comprising an scFv and a second antigen binding domain comprising a Fab or VHH. In some embodiments, the antigen-binding polypeptide constructs comprise a first antigen binding domain and a second antigen binding domain, wherein both antigen binding domains comprise an scFv. In some embodiments, the first and second antigen binding domains each comprise a Fab. In some embodiments, the first and second antigen binding domains each comprise an Fc domain. Any combination of antibody formats is suitable for the bi-specific antibody constructs disclosed herein.

In example embodiments, immune cells can be engaged to tumor cells. In example embodiments, tumor cells are targeted with a bsAb or BiTE having affinity for both the tumor and a payload. In example embodiments, two targets are disrupted on a tumor cell by the bsAb. By means of an example, an agent, such as a bi-specific antibody or BiTE, capable of specifically binding to a gene product expressed on the cell surface of the immune cells (e.g., CD3, CD8, CD28, CD16) and a peptide presented on the surface by a tumor cell (e.g., neoantigens) may be used for targeting polyfunctional immune cells to tumor cells. Immune cells targeted to a tumor may include T cells or Natural Killer cells. By means of an example, an agent, such as a bi-specific antibody or BiTE, capable of specifically binding to a gene product expressed on the cell surface of suppressive immune cells (e.g., Tregs) and a peptide presented on the surface by a cell having an autoimmune reaction may be used for targeting suppressive immune cells to sites of autoimmunity.

### Antibody-Drug-Conjugate

In example embodiments, a binding protein is assembled into an antibody drug conjugate. The term “antibody-drug-conjugate” or “ADC” refers to a binding protein, such as an antibody or antigen binding fragment thereof, chemically linked to one or more chemical drug(s) (also referred to herein as agent(s)) that may optionally be therapeutic or cytotoxic agents. In a preferred embodiment, an ADC includes an antibody, a cytotoxic or therapeutic drug, and a linker that enables attachment or conjugation of the drug to the antibody. An ADC typically has anywhere from 1 to 8 drugs conjugated to the antibody, including drug loaded species of 2, 4, 6, or 8.

In certain embodiments, the ADC specifically binds to a peptide presented on the cell surface of a tumor cell. By means of an example, an agent, such as an antibody, capable of specifically binding to a peptide presented on the cell surface of the tumor cells may be conjugated with a therapeutic or effector agent for targeted delivery of the therapeutic or effector agent to the immune cells.

Examples of such therapeutic or effector agents include immunomodulatory classes as discussed herein, such as without limitation a toxin, drug, radionuclide, cytokine, lymphokine, chemokine, growth factor, tumor necrosis factor, hormone, hormone antagonist, enzyme, oligonucleotide, siRNA, RNAi, photoactive therapeutic agent, anti-angiogenic agent and pro-apoptotic agent.

Non-limiting examples of drugs that may be included in the ADCs are mitotic inhibitors (e.g., maytansinoid DM4), antitumor antibiotics, immunomodulating agents, vectors for gene therapy, alkylating agents, antiangiogenic agents, antimetabolites, boron-containing agents, chemoprotective agents, hormones, antihormone agents, corticosteroids, photoactive therapeutic agents, oligonucleotides, radionuclide agents, topoisomerase inhibitors, tyrosine kinase inhibitors, and radiosensitizers.

Example toxins include ricin, abrin, alpha toxin, saporin, ribonuclease (RNase), DNase I, Staphylococcal enterotoxin-A, pokeweed antiviral protein, gelonin, diphtheria toxin, Pseudomonas exotoxin, or Pseudomonas endotoxin.

Example radionuclides include ^{103m}Rh, ¹⁰³Ru, ¹⁰⁵Rh, ¹⁰⁵Ru, ¹⁰⁷Hg, ¹⁰⁹Pd, ¹⁰⁹Pt, ¹¹¹Ag, ¹¹¹In, ^{113m}In ¹¹⁹Sb, ¹¹C, ^{121m}Te, ^{122m}Te, ¹²⁵I, ^{125m}Te, ¹²⁶I, ¹³¹I, ¹³³I, ¹³N, ¹⁴²Pr, ¹⁴³Pr, ¹⁴⁹Pm, ¹⁵²Dy, ¹⁵³Sm, ¹⁵O, ¹⁶¹Ho, ¹⁶¹Tb, ¹⁶⁵Tm, ¹⁶⁶Dy, ¹⁶⁶Ho, ¹⁶⁷Tm, ¹⁶⁸Tm, ¹⁶⁹Er, ¹⁶⁹Yb, ¹⁷⁷Lu, ¹⁸⁶Re, ¹⁸⁸Re, ^{189m}Os, ¹⁸⁹Re, ¹⁹²Ir, ¹⁹⁴Ir, ¹⁹⁷Pt, ¹⁹⁸Au, ¹⁹⁹Au, ²⁰¹Tl, ²⁰³Hg, ²¹¹At, ²¹¹Bi, ²¹¹Pb, ²¹²Bi, ²¹²Pb, ²¹³Bi, ²¹⁵Po, ²¹⁷At, ²¹⁹Rn, ²²¹Fr, ²²³Ra, ²²⁴Ac, ²²⁵Ac, ²²⁵Fm, ³²P, ³³P, ⁴⁷Sc, ⁵¹Cr, ⁵⁷Co, ⁵⁸Co, ⁵⁹Fe, ⁶²Cu, ⁶⁷Cu, ⁶⁷Ga, ⁷⁵Br, ⁷⁵Se, ⁷⁶Br, ⁷⁷As, ⁷⁷Br, ^{80m}Br, ⁸⁹Sr, ⁹⁰Y, ⁹⁵Ru, ⁹⁷Ru, ⁹⁹Mo or ^{99m}Tc. Preferably, the radionuclide may be an alpha-particle-emitting radionuclide.

Example enzymes include malate dehydrogenase, staphylococcal nuclease, delta-V-steroid isomerase, yeast alcohol dehydrogenase, alpha-glycerophosphate dehydrogenase, triose phosphate isomerase, horseradish peroxidase, alkaline phosphatase, asparaginase, glucose oxidase, beta-galactosidase, ribonuclease, urease, catalase, glucose-6-phosphate dehydrogenase, glucoamylase or acetylcholinesterase. Such enzymes may be used, for example, in combination with prodrugs that are administered in relatively non-toxic form and converted at the target site by the enzyme into a cytotoxic agent. In other alternatives, a drug may be converted into less toxic form by endogenous enzymes in the subject but may be reconverted into a cytotoxic form by the therapeutic enzyme.

### Therapeutic Antibody Modifications

In certain example embodiments, the therapeutic antibodies of the present invention may be modified, such that they acquire advantageous properties for therapeutic use (e.g., stability and specificity), but maintain their biological activity. Therapeutic antibodies may be modified to increase stability or to provide characteristics that improve efficacy of the antibody when administered to a subject in vivo. As used herein in reference to therapeutic antibodies, the terms “modified”, “modification” and the like refer to one or more changes that enhance a desired property of the therapeutic antibody. “Modification” includes a covalent chemical modification that does not alter the primary amino acid sequence of the therapeutic antibody itself. Such desired properties include, for example, prolonging the in vivo half-life, increasing the stability, reducing the clearance, altering the immunogenicity or allergenicity, or cellular targeting. Changes to a therapeutic antibody that may be carried out include, but are not limited to, conjugation to a carrier protein, conjugation to a ligand, conjugation to another antibody, PEGylation, polysialylation HESylation, recombinant PEG mimetics, Fc fusion, albumin fusion, nanoparticle attachment, nanoparticulate encapsulation, cholesterol fusion, iron fusion, acylation, amidation, glycosylation, side chain oxidation, phosphorylation, biotinylation, the addition of a surface active material, the addition of amino acid mimetics, or the addition of unnatural amino acids. Modified therapeutic antibodies also include analogs. By “analog” is meant a molecule that is not identical but has analogous functional or structural features. For example, a therapeutic antibody analog retains the biological activity of a corresponding antibody, while having certain biochemical modifications that enhance the analog's function relative to another antibody. Such biochemical modifications could increase the analog's protease resistance, membrane permeability, or half-life, without altering, for example, antigen binding. An analog may include an unnatural amino acid.

The recitation of a listing of chemical groups in any definition of a variable herein includes definitions of that variable as any single group or combination of listed groups. The recitation of an embodiment for a variable or aspect herein includes that embodiment as any single embodiment or in combination with any other embodiments or portions thereof.

Modified antibodies (e.g., fusion proteins) may include a spacer or a linker. The terms “spacer” or “linker” as used in reference to a fusion protein refers to a peptide that joins the proteins comprising a fusion protein. Generally, a spacer has no specific biological activity other than to join or to preserve some minimum distance or other spatial relationship between the proteins. However, in certain embodiments, the constituent amino acids of a spacer may be selected to influence some property of the molecule such as the folding, net charge, or hydrophobicity of the molecule. Suitable linkers for use in an embodiment of the present invention are well known to those of skill in the art and include, but are not limited to, straight or branched-chain carbon linkers, heterocyclic carbon linkers, or peptide linkers. The linker is used to separate two peptides by a distance sufficient to ensure that, in a preferred embodiment, each peptide properly folds. Preferred peptide linker sequences adopt a flexible extended conformation and do not exhibit a propensity for developing an ordered secondary structure. Typical amino acids in flexible protein regions include Gly, Asn and Ser. Virtually any permutation of amino acid sequences containing Gly, Asn and Ser would be expected to satisfy the above criteria for a linker sequence. Other near neutral amino acids, such as Thr and Ala, also may be used in the linker sequence. Still other amino acid sequences that may be used as linkers are disclosed in Maratea et al. (1985), Gene 40:39-46; Murphy et al. (1986) Proc. Nat'l. Acad. Sci. USA 83:8258-62; U.S. Pat. No. 4,935,233; and U.S. Pat. No. 4,751,180.

The clinical effectiveness of protein therapeutics (e.g., antibodies) is often limited by short plasma half-life and susceptibility to protease degradation. Studies of various therapeutic proteins (e.g., filgrastim) have shown that such difficulties may be overcome by various modifications, including conjugating or linking the polypeptide sequence to any of a variety of non-proteinaceous polymers, e.g., polyethylene glycol (PEG), polypropylene glycol, or polyoxyalkylenes (see, for example, typically via a linking moiety covalently bound to both the protein and the nonproteinaceous polymer, e.g., a PEG).

It is well known that the properties of certain proteins can be modulated by attachment of polyethylene glycol (PEG) polymers, which increases the hydrodynamic volume of the protein and thereby slows its clearance by kidney filtration. (See, e.g., Clark et al., J. Biol. Chem. 271:21969-21977 (1996)). Such PEG-conjugated biomolecules have been shown to possess clinically useful properties, including better physical and thermal stability, protection against susceptibility to enzymatic degradation, increased solubility, longer in vivo circulating half-life and decreased clearance, reduced immunogenicity and antigenicity, and reduced toxicity. Therefore, it is envisioned that certain agents can be PEGylated (e.g., on peptide residues) to provide enhanced therapeutic benefits such as, for example, increased efficacy by extending half-life in vivo. In certain embodiments, PEGylation of the agents may be used to extend the serum half-life of the agents and allow for particular agents to be capable of crossing the blood-brain barrier. Thus, in one embodiment, PEGylating antibodies improve the pharmacokinetics and pharmacodynamics of the antibodies.

In regard to peptide PEGylation methods, reference is made to Lu et al., Int. J. Pept. Protein Res.43: 127-38 (1994); Lu et al., Pept. Res. 6:140-6 (1993); Felix et al., Int. J. Pept. Protein Res. 46:253-64 (1995); Gaertner et al., Bioconjug. Chem. 7:38-44 (1996); Tsutsumi et al., Thromb. Haemost. 77:168-73 (1997); Francis et al., hit. J. Hematol. 68:1-18 (1998); Roberts et al., J. Pharm. Sci. 87:1440-45 (1998); and Tan et al., Protein Expr. Purif. 12:45-52 (1998). Polyethylene glycol or PEG is meant to encompass any of the forms of PEG that have been used to derivatize other proteins, including, but not limited to, mono-(C1-10) alkoxy or aryloxy-polyethylene glycol. Suitable PEG moieties include, for example, 40 kDa methoxy poly(ethylene glycol) propionaldehyde (Dow, Midland, Mich.); 60 kDa methoxy poly(ethylene glycol) propionaldehyde (Dow, Midland, Mich.); 40 kDa methoxy poly(ethylene glycol) maleimido-propionamide (Dow, Midland, Mich.); 31 kDa alpha-methyl-w-(3-oxopropoxy), polyoxyethylene (NOF Corporation, Tokyo); mPEG2-NHS-40k (Nektar); mPEG2-MAL-40k (Nektar), SUNBRIGHT GL2-400MA ((PEG) 240kDa) (NOF Corporation, Tokyo), SUNBRIGHT ME-200MA (PEG20kDa) (NOF Corporation, Tokyo). The PEG groups are generally attached to the peptide (e.g., RBD) via acylation or alkylation through a reactive group on the PEG moiety (for example, a maleimide, an aldehyde, amino, thiol, or ester group) to a reactive group on the peptide (for example, an aldehyde, amino, thiol, a maleimide, or ester group).

The PEG molecule(s) may be covalently attached to any Lys, Cys, or K(CO(CH2)2SH) residues at any position in a peptide. In certain embodiments, the antibodies described herein can be PEGylated directly to any amino acid at the N-terminus by way of the N-terminal amino group. A “linker arm” may be added to a peptide to facilitate PEGylation. PEGylation at the thiol side-chain of cysteine has been widely reported (see, e.g., Caliceti & Veronese, Adv. Drug Deliv. Rev. 55: 1261-77 (2003)). If there is no cysteine residue in the peptide, a cysteine residue can be introduced through substitution or by adding a cysteine to the N-terminal amino acid. In certain embodiments, proteins are PEGylated through the side chains of a cysteine residue added to the N-terminal amino acid.

In exemplary embodiments, the PEG molecule(s) may be covalently attached to an amide group in the C-terminus of a peptide. In certain embodiments, the PEG molecule used in modifying an agent of the present invention is branched while in other embodiments, the PEG molecule may be linear. In particular aspects, the PEG molecule is between 1 kDa and 100 kDa in molecular weight. In further aspects, the PEG molecule is selected from 10, 20, 30, 40, 50, 60, and 80 kDa. In further still aspects, it is selected from 20, 40, or 60 kDa. Where there are two PEG molecules covalently attached to the agent of the present invention, each is 1 to 40 kDa and in particular aspects, they have molecular weights of 20 and 20 kDa, 10 and 30 kDa, 30 and 30 kDa, 20 and 40 kDa, or 40 and 40 kDa. In particular aspects, the antibodies contain mPEG-cysteine. The mPEG in mPEG-cysteine can have various molecular weights. The range of the molecular weight is preferably 5 kDa to 200 kDa, more preferably 5 kDa to 100 kDa, and further preferably 20 kDa to 60 kDA. The mPEG can be linear or branched.

The present disclosure also contemplates the use of PEG Mimetics. Recombinant PEG mimetics have been developed that retain the attributes of PEG (e.g., enhanced serum half-life) while conferring several additional advantageous properties. By way of example, simple polypeptide chains (comprising, for example, Ala, Glu, Gly, Pro, Ser and Thr) capable of forming an extended conformation similar to PEG can be produced recombinantly already fused to the antibodies (e.g., Amunix' XTEN technology; Mountain View, CA). This obviates the need for an additional conjugation step during the manufacturing process. Moreover, established molecular biology techniques enable control of the side chain composition of the polypeptide chains, allowing optimization of immunogenicity and manufacturing properties.

Glycosylation can dramatically affect the physical properties of proteins and can also be important in protein stability, secretion, and subcellular localization (see, e.g., Solá and Griebenow, Glycosylation of Therapeutic Proteins: An Effective Strategy to Optimize Efficacy. BioDrugs. 2010; 24(1): 9-21). Proper glycosylation can be essential for biological activity. In fact, some genes from eukaryotic organisms, when expressed in bacteria (e.g., E. coli) which lack cellular processes for glycosylating proteins, yield proteins that are recovered with little or no activity by virtue of their lack of glycosylation. For purposes of the present disclosure, “glycosylation” is meant to broadly refer to the enzymatic process that attaches glycans to proteins, lipids or other organic molecules. The use of the term “glycosylation” in conjunction with the present disclosure is generally intended to mean adding or deleting one or more carbohydrate moieties (either by removing the underlying glycosylation site or by deleting the glycosylation by chemical and/or enzymatic means), and/or adding one or more glycosylation sites that may or may not be present in the original sequence.

Addition of glycosylation sites can be accomplished by altering the amino acid sequence. The alteration to the polypeptide may be made, for example, by the addition of, or substitution by, one or more serine or threonine residues (for O-linked glycosylation sites) or asparagine residues (for N-linked glycosylation sites). The structures of N-linked and O-linked oligosaccharides and the sugar residues found in each type may be different. One type of sugar that is commonly found on both is N-acetylneuraminic acid (hereafter referred to as sialic acid). Sialic acid is usually the terminal residue of both N-linked and O-linked oligosaccharides and, by virtue of its negative charge, may confer acidic properties to the glycoprotein. A particular embodiment of the present disclosure comprises the generation and use of N-glycosylation variants.

The present disclosure also contemplates the use of polysialylation, the conjugation of peptides and proteins to the naturally occurring, biodegradable a-(2→8) linked polysialic acid (“PSA”) in order to improve their stability and in vivo pharmacokinetics. PSA is a biodegradable, non-toxic natural polymer that is highly hydrophilic, giving it a high apparent molecular weight in the blood which increases its serum half-life. In addition, polysialylation of a range of peptide and protein therapeutics has led to markedly reduced proteolysis, retention of activity in vivo activity, and reduction in immunogenicity and antigenicity (see, e.g., G. Gregoriadis et al., Int. J. Pharmaceutics 300(1-2): 125-30). As with modifications with other conjugates (e.g., PEG), various techniques for site-specific polysialylation are available (see, e.g., T. Lindhout et al., PNAS 108(18)7397-7402 (2011)).

Additional suitable components and molecules for conjugation include, for example, thyroglobulin; albumins such as human serum albumin (HAS); tetanus toxoid; Diphtheria toxoid; polyamino acids such as poly(D-lysine: D-glutamic acid); VP6 polypeptides of rotaviruses; influenza virus hemaglutinin, influenza virus nucleoprotein; Keyhole Limpet Hemocyanin (KLH); and hepatitis B virus core protein and surface antigen; or any combination of the foregoing.

Fusion of albumin to one or more antibodies of the present disclosure can, for example, be achieved by genetic manipulation, such that the DNA coding for HSA, or a fragment thereof, is joined to the DNA coding for the one or more antibodies. Albumin itself may be modified to extend its circulating half-life. Fusion of the modified albumin to one or more polypeptides can be attained by the genetic manipulation techniques described above or by chemical conjugation; the resulting fusion molecule has a half-life that exceeds that of fusions with non-modified albumin. (See WO2011/051489).

Several albumin-binding strategies have been developed as alternatives for direct fusion, including albumin binding through a conjugated fatty acid chain (acylation). Because serum albumin is a transport protein for fatty acids, these natural ligands with albumin-binding activity have been used for half-life extension of small protein therapeutics. For example, (LEVEMIR), an approved product for diabetes, comprises a myristyl chain conjugated to a genetically-modified insulin, resulting in a long-acting insulin analog.

Another type of modification is to conjugate (e.g., link) one or more additional components or molecules at the N- and/or C-terminus of a polypeptide sequence, such as another protein, or a carrier molecule. Thus, an exemplary polypeptide sequence can be provided as a conjugate with another component or molecule. A conjugate modification may result in a polypeptide sequence that retains activity with an additional or complementary function or activity of the second molecule. For example, a polypeptide sequence may be conjugated to a molecule, e.g., to facilitate solubility, storage, in vivo or shelf half-life or stability, reduction in immunogenicity, delayed or controlled release in vivo, etc. Other functions or activities include a conjugate that reduces toxicity relative to an unconjugated polypeptide sequence, a conjugate that targets a type of cell or organ more efficiently than an unconjugated polypeptide sequence, or a drug to further counter the causes or effects associated with a disorder or disease as set forth herein.

The present disclosure contemplates the use of other modifications, currently known or developed in the future, of the polypeptides to improve one or more properties. One such method for prolonging the circulation half-life, increasing the stability, reducing the clearance, or altering the immunogenicity or allergenicity of a polypeptide of the present disclosure involves modification of the polypeptide sequences by hesylation, which utilizes hydroxyethyl starch derivatives linked to other molecules in order to modify the molecule's characteristics. Various aspects of hesylation are described in, for example, U.S. Patent Appln. Nos. 2007/0134197 and 2006/0258607.

In particular embodiments, the antibodies include a protecting group covalently joined to the N-terminal amino group. In exemplary embodiments, a protecting group covalently joined to the N-terminal amino group of the proteins reduces the reactivity of the amino terminus under in vivo conditions. Amino protecting groups include —C1-10 alkyl, —C1-10 substituted alkyl, —C2-10 alkenyl, —C2-10 substituted alkenyl, aryl, —C1-6 alkyl aryl, —C(O)—(CH2)1-6—COOH, —C(O)—C1-6 alkyl, —C(O)-aryl, —C(O)—O—C1-6 alkyl, or —C(O)—O-aryl. In particular embodiments, the amino terminus protecting group is selected from the group consisting of acetyl, propyl, succinyl, benzyl, benzyloxycarbonyl, and t-butyloxycarbonyl. In other embodiments, deamination of the N-terminal amino acid is another modification that may be used for reducing the reactivity of the amino terminus under in vivo conditions.

Chemically modified compositions of the antibodies wherein the antibody is linked to a polymer are also included within the scope of the present invention. The polymer selected is usually modified to have a single reactive group, such as an active ester for acylation or an aldehyde for alkylation, so that the degree of polymerization may be controlled. Included within the scope of polymers is a mixture of polymers. Preferably, for therapeutic use of the end-product preparation, the polymer will be pharmaceutically acceptable. The polymer or mixture thereof may include but is not limited to polyethylene glycol (PEG), monomethoxy-polyethylene glycol, dextran, cellulose, or other carbohydrate-based polymers, poly-(N-vinyl pyrrolidone) polyethylene glycol, propylene glycol homopolymers, a polypropylene oxide/ethylene oxide co-polymer, polyoxyethylated polyols (for example, glycerol), and polyvinyl alcohol.

In other embodiments, the antibodies are modified by PEGylation, cholesterylation, or palmitoylation. The modification can be to any amino acid residue. In preferred embodiments, the modification is to the N-terminal amino acid of the antibodies, either directly to the N-terminal amino acid or by way coupling to the thiol group of a cysteine residue added to the N-terminus or a linker added to the N-terminus such as trimesoyl tris(3,5-dibromosalicylate (Ttds). In certain embodiments, the N-terminus of the antibodies comprise a cysteine residue to which a protecting group is coupled to the N-terminal amino group of the cysteine residue and the cysteine thiolate group is derivatized with N-ethylmaleimide, PEG group, cholesterol group, or palmitoyl group. In other embodiments, an acetylated cysteine residue is added to the N-terminus of the agents, and the thiol group of the cysteine is derivatized with N-ethylmaleimide, PEG group, cholesterol group, or palmitoyl group. In certain embodiments, the antibodies of the present invention consist of an amino acid sequence which is bound with a methoxypolyethylene glycol(s) via a linker.

Substitutions of amino acids may be used to modify an antibody of the present invention. The phrase “substitution of amino acids” as used herein encompasses substitution of amino acids that are the result of both conservative and non-conservative substitutions. Conservative substitutions are the replacement of an amino acid residue by another similar residue in a polypeptide. Typical but not limiting conservative substitutions are the replacements, for one another, among the aliphatic amino acids Ala, Val, Leu and Ile; interchange of Ser and Thr containing hydroxy residues, interchange of the acidic residues Asp and Glu, interchange between the amide-containing residues Asn and Gln, interchange of the basic residues Lys and Arg, interchange of the aromatic residues Phe and Tyr, and interchange of the small-sized amino acids Ala, Ser, Thr, Met, and Gly. Non-conservative substitutions are the replacement, in a polypeptide, of an amino acid residue by another residue which is not biologically similar. For example, the replacement of an amino acid residue with another residue that has a substantially different charge, a substantially different hydrophobicity, or a substantially different spatial configuration.

One of skill in the art from this disclosure and the knowledge in the art will appreciate that there are a variety of ways in which to produce such therapeutic antibodies. In general, such therapeutic antibodies may be produced either in vitro or in vivo. Therapeutic antibodies may be produced in vitro as peptides or polypeptides, which may then be formulated into a pharmaceutical composition and administered to a subject. Such in vitro production may occur by a variety of methods known to one of skill in the art such as, for example, peptide synthesis or expression of a peptide/polypeptide from a DNA or RNA molecule in any of a variety of bacterial, eukaryotic, or viral recombinant expression systems, followed by purification of the expressed antibodies (e.g., with protein A or G). Alternatively, antibodies may be produced in vivo by introducing molecules (e.g., DNA, RNA, viral expression systems, and the like) that encode antibodies into a subject, whereupon the encoded therapeutic antibodies are expressed.

In certain embodiments, the antibodies as defined for the present invention include derivatives that are modified, i.e., by the covalent attachment of any type of molecule to the antibody such that covalent attachment does not prevent the antibody from generating an anti-idiotypic response. For example, but not by way of limitation, the antibody derivatives include antibodies that have been modified, e.g., by glycosylation, acetylation, pegylation, phosphylation, amidation, derivatization by known protecting/blocking groups, proteolytic cleavage, linkage to a cellular ligand or other protein, etc. Any of numerous chemical modifications may be carried out by known techniques, including, but not limited to, specific chemical cleavage, acetylation, formylation, metabolic synthesis of tunicamycin, etc. Additionally, the derivative may contain one or more non-classical amino acids.

Simple binding assays can be used to screen for or detect antibodies that bind to a target protein, or disrupt the interaction between proteins (e.g., a receptor and a ligand). Because certain targets of the present invention are transmembrane proteins, assays that use the soluble forms of these proteins rather than full-length protein can be used, in some embodiments. Soluble forms include, for example, those lacking the transmembrane domain and/or those comprising the IgV domain or fragments thereof which retain their ability to bind their cognate binding partners. Further, agents that inhibit or enhance protein interactions for use in the compositions and methods described herein, can include recombinant peptido-mimetics.

Detection methods useful in screening assays include antibody-based methods, detection of a reporter moiety, detection of cytokines as described herein, and detection of a gene or gene signature.

Another variation of assays to determine binding of a receptor protein to a ligand protein is through the use of affinity biosensor methods. Such methods may be based on the piezoelectric effect, electrochemistry, or optical methods, such as ellipsometry, optical wave guidance, and surface plasmon resonance (SPR).

### Administration of Therapeutic Antibodies

For therapeutic uses, the antibodies described herein may be administered systemically, for example, formulated in a pharmaceutically-acceptable buffer such as physiological saline. Preferable routes of administration include, for example, subcutaneous, intravenous, interperitoneal, intramuscular, or intradermal injections that provide continuous, sustained levels of the antibody in the patient. Treatment of human patients or other animals will be carried out using a therapeutically effective amount of a therapeutic identified herein in a physiologically-acceptable carrier. Suitable carriers and their formulation are described, for example, in Remington's Pharmaceutical Sciences by E. W. Martin. The amount of the therapeutic agent to be administered varies depending upon the manner of administration, the age and body weight of the patient, and with the clinical symptoms of the neoplasia. Generally, amounts will be in the range of those used for other agents used in the treatment of other diseases associated with neoplasia, although in certain instances lower amounts will be needed because of the increased specificity of the compound. For example, a therapeutic compound is administered at a dosage that is cytotoxic to a neoplastic cell.

Human dosage amounts can initially be determined by extrapolating from the amount of antibody used in mice, as a skilled artisan recognizes it is routine in the art to modify the dosage for humans compared to animal models. Of course, this dosage amount may be adjusted upward or downward, as is routinely done in such treatment protocols, depending on the results of the initial clinical trials and the needs of a particular patient.

The therapeutic regimens disclosed herein comprise administration of antibodies of the invention or pharmaceutical compositions thereof to the patient in a single dose or in multiple doses (e.g., 1, 2, 3, 4, 5, 6, 7, 8, 10, 15, 20, or more doses). In one aspect, the therapeutic regimens comprise administration of the antibodies of the invention or pharmaceutical compositions thereof in multiple doses. When administered in multiple doses, the antibodies are administered with a frequency and in an amount sufficient to treat SARS-COV-2. For example, the frequency of administration ranges from once a day up to about four times a day. In another example, the frequency of administration ranges from about once a week up to about once every six weeks.

Significant progress has been made in understanding pharmacokinetics (PK), pharmacodynamics (PD), as well as toxicity profiles of therapeutic antibodies in animals and humans, which have been in commercial development for more than three decades (see, e.g., Vugmeyster et al., Pharmacokinetics and toxicology of therapeutic proteins: Advances and challenges, World J Biol Chem. 2012 Apr. 26; 3(4): 73-92). In certain embodiments, therapeutic antibodies are administered by parenteral routes, such as intravenous (IV), subcutaneous (SC) or intramuscular (IM) injection. Molecular size, hydrophilicity, and gastric degradation are the main factors that preclude gastrointestinal (GI) absorption of therapeutic proteins (see, e.g., Keizer, et al., Clinical pharmacokinetics of therapeutic monoclonal antibodies. Clin Pharmacokinet. 2010 August; 49(8):493-507). Pulmonary delivery with aerosol formulations or dry powder inhalers has been used for selected proteins, e.g., exubera (TM) (see, e.g., Scheuch and Siekmeier, Novel approaches to enhance pulmonary delivery of proteins and peptides. J Physiol Pharmacol. 2007 November; 58 Suppl 5(Pt 2):615-25). Intravitreal injections have been used for peptides and proteins that require only local activity (see, e.g., Suresh, et al., Ocular Delivery of Peptides and Proteins. In: Van Der Walle C., editor. Peptide and Protein Delivery. London: Academic Press; 2011. pp. 87-103). In certain embodiments, SC administration of therapeutic antibodies is often a preferred route. In particular, the suitability of SC dosing for self-administration translates into significantly reduced treatment costs.

The pharmaceutical composition may be administered parenterally by injection, infusion or implantation (subcutaneous, intravenous, intramuscular, intraperitoneal, or the like) in dosage forms, formulations, or via suitable delivery devices or implants containing conventional, non-toxic pharmaceutically acceptable carriers and adjuvants. The formulation and preparation of such compositions are well known to those skilled in the art of pharmaceutical formulation. Formulations can be found in Remington: The Science and Practice of Pharmacy, supra.

As indicated above, the pharmaceutical compositions according to the invention may be in the form suitable for sterile injection. To prepare such a composition, the suitable antibodies are dissolved or suspended in a parenterally acceptable liquid vehicle. Among acceptable vehicles and solvents that may be employed are water, water adjusted to a suitable pH by addition of an appropriate amount of hydrochloric acid, sodium hydroxide or a suitable buffer, 1,3-butanediol, Ringer's solution, and isotonic sodium chloride solution and dextrose solution. The aqueous formulation may also contain one or more preservatives (e.g., methyl, ethyl or n-propyl p-hydroxybenzoate).

### Administration of Cells

The administration of cells or population of cells, such as immune system cells or cell populations, such as more particularly immunoresponsive cells or cell populations, as disclosed herein may be carried out in any convenient manner, including by aerosol inhalation, injection, ingestion, transfusion, implantation, or transplantation. The cells or population of cells may be administered to a patient subcutaneously, intradermally, intratumorally, intranodally, intramedullary, intramuscularly, intrathecally, by intravenous or intralymphatic injection, or intraperitoneally. In some embodiments, the disclosed CARs may be delivered or administered into a cavity formed by the resection of tumor tissue (i.e., intracavity delivery) or directly into a tumor prior to resection (i.e., intratumoral delivery). In one embodiment, the cell compositions of the present invention are preferably administered by intravenous injection.

The administration of the cells or population of cells can consist of the administration of 10⁴-10⁹ cells per kg body weight, preferably 10⁵ to 10⁶ cells/kg body weight including all integer values of cell numbers within those ranges. Dosing in CAR T cell therapies may for example involve administration of from 10⁶ to 10⁹ cells/kg, with or without a course of lymphodepletion, for example with cyclophosphamide. The cells or population of cells can be administrated in one or more doses. In another embodiment, the effective amount of cells are administrated as a single dose. In another embodiment, the effective amount of cells are administrated as more than one dose over a period time. Timing of administration is within the judgment of managing physician and depends on the clinical condition of the patient. The cells or population of cells may be obtained from any source, such as a blood bank or a donor. While individual needs vary, determination of optimal ranges of effective amounts of a given cell type for a particular disease or conditions are within the skill of one in the art. An effective amount means an amount which provides a therapeutic or prophylactic benefit. The dosage administrated will be dependent upon the age, health and weight of the recipient, kind of concurrent treatment, if any, frequency of treatment and the nature of the effect desired.

In another embodiment, the effective amount of cells or composition comprising those cells are administrated parenterally. The administration can be an intravenous administration. The administration can be directly done by injection within a tumor.

Further embodiments are illustrated in the following Examples which are given for illustrative purposes only and are not intended to limit the scope of the invention.

### EXAMPLES

### Example 1—Selection of TCR-Mimetic Binding Proteins Using Ribosome Display

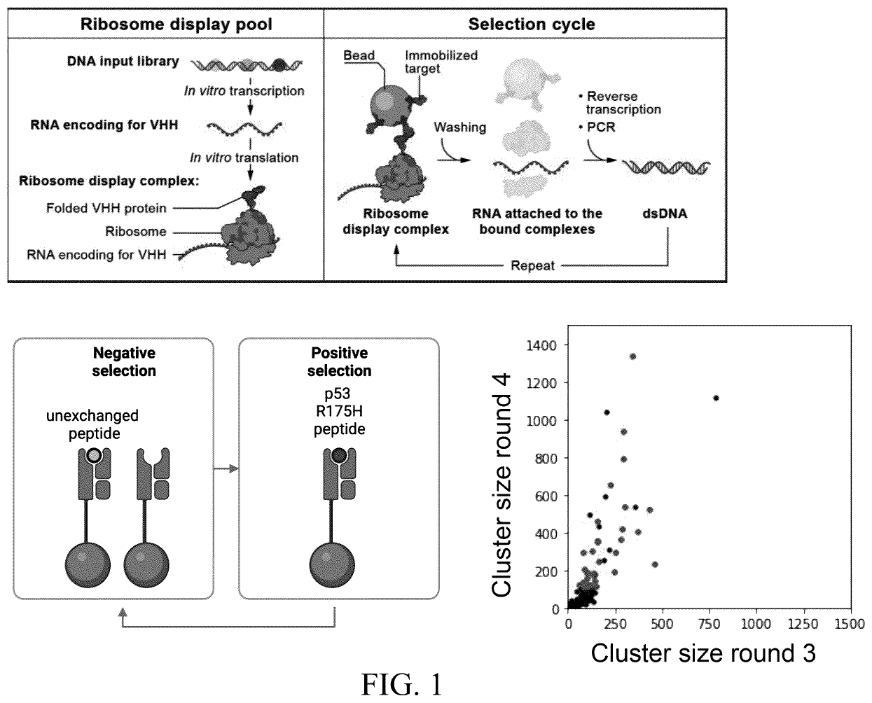

Applicants have developed a set of methods for selecting binding proteins, including antibodies and non-antibody scaffolds such as nanobodies (used as an example below), against peptide-MHC complexes for the high-throughput discovery of TCR-mimetic binding proteins. The methods utilize a ribosome display platform to rapidly (within 24 hours) select thousands of candidates from libraries containing up to 10¹² protein sequences (FIG. 1). In a prototypical process, ribosome display nanobody libraries are expressed via cell-free IVTT and incubated with (1) magnetic beads coated with both pMHC loaded with a control peptide and unloaded MHC to deplete off-target binders (negative selection), followed by (2) magnetic beads coated with pMHC loaded with target peptide (positive selection). After stringent washing the selected protein sequences are recovered via RT-PCR, and this process is repeated for multiple rounds to enrich for high-affinity binders. DNA from the final round of RT-PCR is analyzed via next-generation sequencing; nanobodies containing the same complementarity determining regions (CDRs) are clustered, and representative sequences from the largest clusters are selected as candidates for further analysis. Finally, candidate sequences are synthesized as double-stranded DNA for use in downstream assays (e.g., cloning into a vector).

### Example 2—Validation of TCR-Mimetic Binding Proteins

Binding assay against purified pMHC using E. coli expressed nanobodies and ELISA

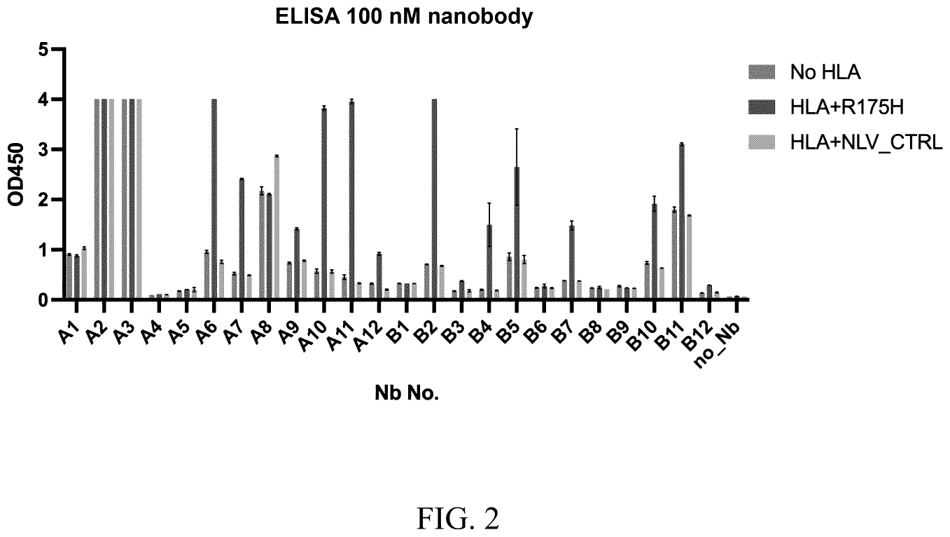

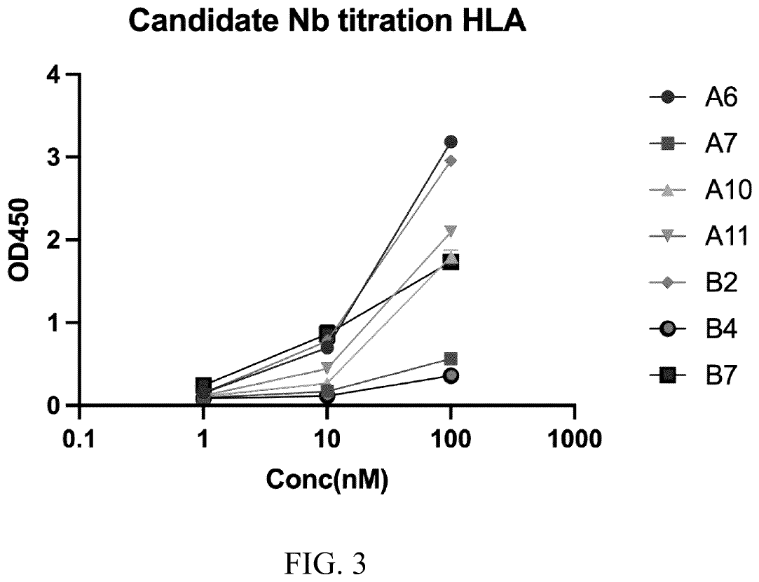

In one embodiment of the process, nanobody sequences are cloned into expression vectors and expressed and purified in E. coli. Purified nanobodies are assayed via ELISA to determine binding to uncoated plate, pMHC loaded with target peptide or control peptides (FIG. 2). In an example embodiment, peptides mutated at one or more residues are used. Nanobodies with specific binding to target peptides can be further assayed to determine binding affinity (EC50) using ELISA (FIG. 3).

### Binding Assay Against Cellular PMHC Using Nanobody Multimers

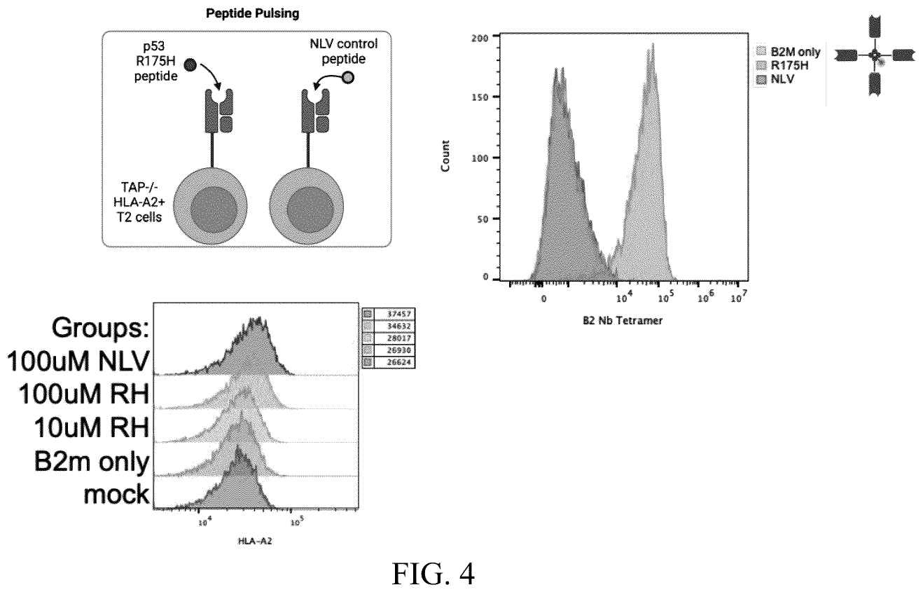

In another embodiment of the process, nanobody sequences are cloned into expression vectors, expressed and purified, biotinylated in vitro, and tetramerized by the addition of streptavidin. Targets displaying target or control peptide on expressed pMHC are produced by pulsing TAP−/− cells with peptide, and nanobody tetramer binding is analyzed with flow cytometry to determine on-target and off-target binding (FIG. 4).

### Binding Assay Against Purified PMHC or Cellular PMHC Using IVTT Expressed Self-Assembled Nanobody Multimers

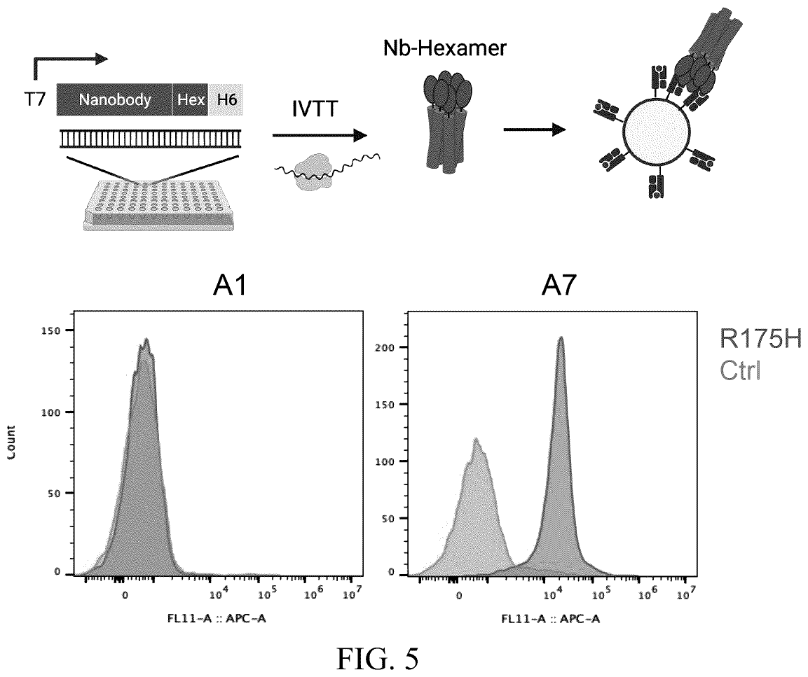

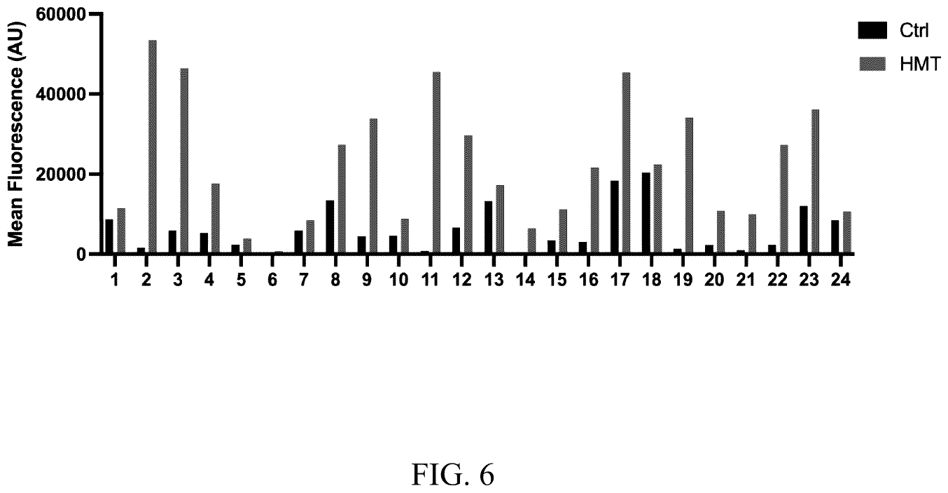

In another embodiment of the process, a multimerizing peptide sequence is appended to the nanobody sequence during DNA synthesis or post-synthesis via PCR. Sequences for the T7 promoter and a terminator sequence are also appended via PCR, and an IVTT reaction is used to produce self-assembled nanobody multimers (FIG. 5). Nanobody multimers are subsequently used to stain bead-bound pMHC or cellular targets loaded with target or control peptide, and nanobody multimer binding is analyzed with flow cytometry to determine on-target and off-target binding (FIG. 6, 7).

### Cellular Libraries of Expressed PMHC

In one embodiment, target cells expressing pMHC complex loaded with target or control peptide are produced by transducing TAP−/− cells with a vector encoding an antigen peptide directly fused to an ER targeting signal peptide. In another embodiment, target cells are produced by expressing single-chain pMHC consisting of (1) target or control peptides, (2) beta-2-microglobulin (B2M), and (3) an HLA allele connected by flexible peptide linkers.

### Nanobody BITE and CAR-T Assays Against Cellular Targets

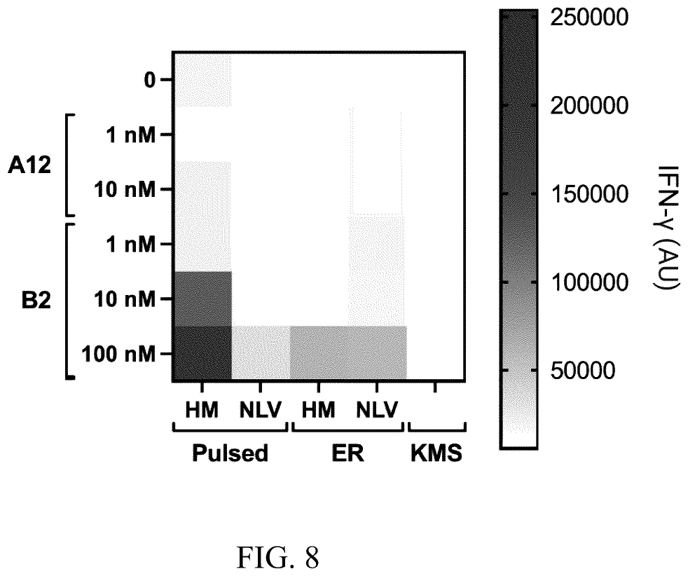

In one embodiment of the process, the activity of candidate nanobodies against pMHC presented on target cells can be evaluated by generating bispecific T cell engager (BITE) proteins consisting of (1) a protein domain binding to CD3, (2) a flexible linker, and (3) the candidate nanobody, and incubating these BITES with CD8+ T cells and target cells encompassing (1) peptide-pulsed TAP−/− cells, (2) TAP−/− cells expressing peptides fused to an ER signal sequence, (3) cells transduced with single-chain pMHC, or (4) cells expressing proteins containing the antigen peptide and processed naturally via the native antigen presentation pathway, including tumor cell lines and patient-derived cells. CD8+ T cell activity is determined by directly evaluating cytotoxicity or by analyzing cytokines released by T cells (FIG. 8).

Various modifications and variations of the described methods, pharmaceutical compositions, and kits of the invention will be apparent to those skilled in the art without departing from the scope and spirit of the invention. Although the invention has been described in connection with specific embodiments, it will be understood that it is capable of further modifications and that the invention as claimed should not be unduly limited to such specific embodiments. Indeed, various modifications of the described modes for carrying out the invention that are obvious to those skilled in the art are intended to be within the scope of the invention. This application is intended to cover any variations, uses, or adaptations of the invention following, in general, the principles of the invention and including such departures from the present disclosure come within known customary practice within the art to which the invention pertains and may be applied to the essential features hereinbefore set forth.

What is claimed is:

1. A method of generating binding proteins with specificity for a target peptide-MHC (pMHC) complex comprising:
a. performing ribosome display using a ribosome display library that generates a set of ribonucleoprotein complexes, each ribonucleoprotein complex comprising a candidate binding polypeptide and RNA encoding the candidate binding polypeptide;
b. negatively selecting ribonucleoprotein complexes displaying off-target binding polypeptides via binding with one or more of a control pMHC, control peptide, and unloaded MHC immobilized on one or more solid supports;
c. positively selecting ribonucleoprotein complexes displaying on-target candidate binding polypeptides via binding with a target pMHC immobilized on one or more solid supports;
d. recovering RNAs encoding the positively selected on-target candidate binding polypeptides;
e. optionally, repeating steps (a) to (d) based on the recovered RNAs in step (d) as the input for a new ribosome display library in step (a); and
f. sequencing the recovered RNAs to identify a final set of on-target binding polypeptides.

2. The method of claim 1, wherein the binding proteins encoded for in the ribosome display library are selected from the group consisting of a nanobody (VHH), antibody fragment (Fab), single-chain variable fragment (scFv), and non-antibody scaffold.

3. The method of claim 1 or 2, further comprising clustering the binding protein sequences containing similar binding domains.

4. The method of claim 3, wherein the binding proteins are clustered based on similarity of complementary determining regions (CDRs).

5. The method of claim 4, wherein the binding proteins clustered contain one or more of the same CDR.

6. The method of any of claims 3 to 5, wherein binding proteins are selected from one or more of the clusters having the largest number of members.

7. The method of any of claims 1 to 6, wherein steps (a) to (d) are repeated more than 1, 2, 3, 4, 5, 6, 7, 8, 9, or 10 rounds.

8. The method of any of claims 1 to 7, further comprising mutagenizing the binding proteins before repeating steps (a) to (d).

9. The method of claim 8, wherein CDRs are randomized.

10. The method of any of claims 1 to 9, wherein the MHC molecule is a class I MHC or class II MHC.

11. The method of claim 10, wherein the MHC molecule is a human HLA allele.

12. The method of any of claims 1 to 11, wherein the one or more solid supports are beads.

13. The method of claim 12, wherein the beads are magnetic beads.

14. The method of any of claims 1 to 13, wherein the target peptide originates from a protein selected from the group consisting of tumor associated antigens, tumor specific antigens, neoantigens, self-antigens, allergens, or pathogen antigens.

15. The method of claim 14, wherein the neoantigens are derived from somatic mutations, RNA splicing, RNA editing, and/or neo-ORFs.

16. The method of any of claims 1 to 15, wherein the target pMHC is specific to a subject's MHC alleles and peptides capable of being presented by the subject's MHC alleles.

17. The method of any of claims 1 to 15, wherein the target pMHC is a pMHC isolated from a target cell.

18. The method of claim 17, wherein the target cell is obtained from a subject.

19. The method of claim 17, wherein the target cell is a cell line.

20. The method of any of claims 17 to 19, wherein the target cell is a tumor cell.

21. The method of any of claims 17 to 19, wherein the target cell is a cell targeted by an autoimmune response.

22. The method of claim 19, wherein the target cell is a cell line that is monoallelic for an MHC molecule.

23. The method of any of claims 17 to 19, wherein the target cell is an antigen presenting cell.

24. The method of any of claims 1 to 13, wherein the control peptide originates from a self-protein.

25. The method of any of the preceding claims, further comprising assembling a selected binding protein into a chimeric antigen receptor (CAR), bispecific engager molecule, diabody, triabody, tetrabody, or minibody.

26. The method of any of the preceding claims, further comprising validating the binding and activity of a selected binding protein.

27. The method of claim 26, wherein a binding protein representative of a cluster is validated.

28. The method of claim 26 or 27, wherein validating comprises expressing a recombinant binding protein and performing an ELISA against purified target pMHC.

29. The method of claim 26 or 27, wherein validating comprises expressing a recombinant binding protein and performing flow cytometry with beads coated with target pMHC or control pMHC.

30. The method of claim 26 or 27, wherein validating comprises expressing a recombinant binding protein and performing flow cytometry with cells displaying the target peptide or control peptide on expressed pMHC.

31. The method of claim 30, wherein the cells are TAP−/− cells.

32. The method of any of claims 28 to 30, wherein the recombinant binding protein is multimerized.

33. The method of claim 32, wherein the recombinant binding protein is biotinylated and tetramerized by the addition of streptavidin.

34. The method of claim 32, wherein the recombinant binding protein is fused to a multimerizing peptide sequence and self-assembled multimers are produced by in vitro transcription/translation (IVTT).

35. The method of claim 26 or 27, wherein validating comprises expressing a recombinant binding protein as a BITE or CAR T cell and performing a T cell cytotoxicity assay.

36. A method of characterizing a peptide-MHC targeting binding protein comprising any method of validating the binding and activity of binding proteins according to any of claims 28 to 35.

37. A cell library comprising a population of cells expressing a plurality of pMHCs, each cell expressing a recombinant vector encoding a peptide, wherein each cell expresses a pMHC complex loaded with a peptide encoded for by the vector.

38. The cell library of claim 37, wherein each vector encodes for a peptide fused to an ER targeting signal peptide.

39. The cell library of claim 37, wherein each vector encodes for a single-chain pMHC comprising a peptide, beta-2-microglobulin (B2M), and an HLA allele connected by flexible peptide linkers.

40. The cell library of any of claims 37 to 39, wherein the cells in the library are TAP−/− cells.

41. A method of characterizing a binding protein comprising incubating the binding protein with a cell library according to any of claims 37 to 40; isolating bound cells; and quantifying the peptides encoded for by the vectors in the isolated cells.
